(19) United States
(12) Patent Application Publication
(10) Pub. No.: US 2005/0112616 A1
(43) Pub. Date: May 26, 2005

(54) Functionalized materials and libraries thereof

(76) Inventors: William Lee, Cambridge, MA (US); Kyoichi Saito, Tokyo (JP)

Correspondence Address: FOLEY & LARDNER LLP, 111 HUNTINGTON AVENUE, 26TH FLOOR, BOSTON, MA, 02199-7610, US

(21) Appl. No.: 10/864,705

(22) Filed: Jun. 9, 2004

Related U.S. Application Data
(63) Continuation of application No. PCT/US02/33942, filed on Oct. 23, 2002.
Application No. PCT/US02/33942 is a continuation-in-part of application No. 10/126,297, filed on Apr. 19, 2002.
(60) Provisional application No. 60/339,949, filed on Dec. 10, 2001.
(60) Provisional application No. 60/339,951, filed on Dec. 10, 2001.
(60) Provisional application No. 60/347,535, filed on Jan. 11, 2002.
(60) Provisional application No. 60/347,542, filed on Jan. 11, 2002.
(60) Provisional application No. 60/347,547, filed on Jan. 11, 2002.
(60) Provisional application No. 60/347,602, filed on Jan. 11, 2002.

(51) Int. Cl. B05D 3/00; C12Q 1/68; G01N 33/53; C12M 1/34

(52) U.S. Cl. 435/6; 435/7.1; 435/287.2; 436/518; 427/2.11

(57) ABSTRACT

Compositions are provided herein comprising a material having engrafted polymer brushes. The polymer brushes further comprise one or more functional groups immobilized along the surface of the brushes in a plurality of layers, which confer functional properties to the material compositions. These materials are useful in material libraries for high throughput separation studies, and for the isolation or transfection of microorganisms and cells.

### FIELD OF THE INVENTION

The present invention relates to functionalized materials and libraries thereof, for the separation, purification, concentration, immobilization and synthesis of compounds, as well as applications for using the same.

### BACKGROUND OF THE INVENTION

Isolation and purification of a target molecule is a prerequisite to its study and use, for example, the ability to isolate and identify disease causing microorganisms allows for accurate diagnosis and treatment of disease states, or isolation of a nucleic acid is the first step in the sequencing of the polynucleotide or the polypeptide sequence encoded by a nucleic acid, or the determination of the crystal structure of a protein. There are many methods for isolating, purifying, and concentrating molecules, but the compositions for performing such methods do not have broad application, and are usually applicable to the purification of specific molecules. There remains a need in the art for improved compositions and methods of isolating and concentrating molecules.

### SUMMARY OF THE INVENTION

In general, the invention is based on the discovery that certain materials can be fabricated into compositions that have side chains or polymeric molecular “brushes” which have particular properties, for example, length, thickness, morphology and density. The materials are highly effective for separating, purifying, concentrating and/or immobilizing compounds in a three dimensional conformation, and for synthesizing or otherwise modifying compounds immobilized thereto. The compositions of the present invention are useful in applications that require a high convective flow rate across the material, or are subjected to harsh chemicals, or extreme temperature variations.

In one aspect, the invention includes a substrate material having polymer brushes on at least a first and a second surface, wherein the polymer brushes on the first surface further comprise a first set of functional groups having a charge, and the polymer brushes on the second surface further comprise a second set of functional groups having an opposite charge to the first set of functional groups. In one embodiment, the polymer brushes are formed by radical induced polymerization of the substrate material and the degree of grafting of the polymer brushes is greater than 15%. In another embodiment, the degree of grafting of the polymer brushes is greater than 85%. In yet another embodiment, the material is bipolar and is capable of dissociating water into H⁺ and OH⁻ when a voltage is applied.

In another aspect, the invention includes a method of making a bipolar device by obtaining a material, forming polymer brushes on the material by graft induced polymerization on at least a first and second surface, immobilizing a first functional group to the polymer brushes on the first surface, wherein the first functional group has a charge, and immobilizing a second set of functional groups to the polymer brushes on the second surface, wherein the second functional group has a charge opposite to that of the first functional group. In one embodiment, the polymer brushes have a degree of grafting greater than 15%. In another embodiment, the polymer brushes have a degree of grafting greater than 85%. In another aspect, the invention includes a method of using the bipolar device, to produce a compound, for example, salicylic acid. In one embodiment, the invention includes a method of using the bipolar material to produce acid or alkali from a salt solution. In another embodiment, the material is used for an electrodialysis reaction.

In another aspect, the invention includes a device having a plurality of functionalized materials, further including a substrate material having at least one surface having polymer brushes formed thereon, the polymer brushes presented in a plurality of domains, and one or more functional groups, the functional groups immobilized to the polymer brushes at one or more domains. In one embodiment, the polymer brushes at a plurality of domains are of different morphologies or lengths. In another embodiment, the polymer brushes at a plurality of domains are formed from one or more types of reactive monomers. In yet another embodiment, the polymer brushes at a plurality of domains have a degree of grafting from about 10% to about 500%. In still another embodiment, at least one functional group is immobilized at each domain. In even another embodiment, at least two functional groups are immobilized at each domain. In one embodiment, the functional groups immobilized to the polymer brushes bind one or more targets, including polynucleotide targets, polypeptide targets, polysaccharide targets, lipid targets, organelles, cellular membranes and cell targets, including animal cells, mammalian cells, human cells, fungal cells, viral cells and bacterial cells such as pathogenic bacterial strains e.g., Staphylococcus, Clostridium, Bacillus, and the like. In one embodiment, the functional groups include immunoglobulins or antigen binding fragments thereof. In another embodiment, the functional groups immobilize one or more targets to at least one domain. In yet another embodiment, the functional groups catalyze a reaction involving a target. In one embodiment, the functional groups are charged. In still another embodiment, the functional groups are enzymes, such as restriction enzymes, proteases, kinases or phosphatases. In another embodiment, the functional groups include microdelivery functional groups having a compound. In this embodiment, a target cell is contacted to microdelivery functional groups at one or more domains, thereby causing the target cell to uptake compounds contained in the microdelivery functional groups. Compounds that can be delivered to a cell target through microdelivery groups include, for example but not limited to, short interfering RNA (siRNA), antisense nucleic acids, transposable elements and nucleic acids with similar integration sequences, vectors, proteins and drugs, among others.

In still another aspect, the invention includes a method of making a library of functionalized materials wherein a substrate material is obtained, polymer brushes are formed on the material in a plurality of domains, and at least one functional group is immobilized to the polymer brushes at one or more domains. In one embodiment, the polymer brushes at a plurality of domains are of different morphologies or lengths. In another embodiment, the polymer brushes at a plurality of domains are formed from one or more types of reactive monomers. In yet another embodiment, the polymer brushes at a plurality of domains have a degree of grafting from about 10% to about 500%. In still another embodiment, at least one functional group is immobilized at each domain. In even another embodiment, at least two functional groups are immobilized at each domain. In one embodiment, the functional groups immobilized to the polymer brushes bind one or more targets, including polynucleotide targets, polypeptide targets, polysaccharide targets, lipid targets, organelle targets, cellular membrane targets and cell targets, including animal cells, mammalian cells, human cells, fungal cells, viral cells and bacterial cells such as pathogenic bacterial strains e.g., Staphylococcus, Clostridium, Bacillus, and the like. In one embodiment, the functional groups include immunoglobulins or antigen binding fragments thereof. In another embodiment, the functional groups immobilize one or more targets to at least one domain. In yet another embodiment, the functional groups catalyze a reaction involving a target. In one embodiment, the functional groups are charged. In still another embodiment, the functional groups are enzymes, such as restriction enzymes, proteases, kinases or phosphatases. In another embodiment, the functional groups include microdelivery functional groups having a compound. In this embodiment, a target cell is contacted to microdelivery functional groups at one or more domains, thereby causing the target cell to uptake compounds contained in the microdelivery functional groups.

In one aspect, the invention includes methods of using the devices described to introduce a compound to a target. In one embodiment, the functional groups comprise microdelivery groups containing a compound. In another embodiment, the compound is a nucleic acid, and a cell target is contacted at one or more domains, thereby delivering the nucleic acid to the cell. In yet another embodiment, the compound is a polypeptide, and a cell target is contacted at one or more domains, thereby delivering the polypeptide to the cell. In still another embodiment, the compound is a drug, and a cell target is contacted at one or more domains, thereby delivering the drug to the cell. In one embodiment, the polymer brushes at a plurality of domains are of different morphologies or lengths, and have a degree of grafting from about 10% to about 500%. In another embodiment, the invention includes contacting the domains of the library with a solution comprising a target, and detecting an interaction with the target at one or more of the domains. In one embodiment, the interaction between the target and a domain is detected by measuring radioactive emissions at the domain. In another embodiment, the interaction between the target and a domain is detected by measuring luminescence at the domain. In still another embodiment, the interaction between the target and a domain is detected by measuring fluorescence at the domain. In even another embodiment, the fluorescence measured is generated by fluorescence resonance energy transfer pairs.

In even another aspect, the invention includes a system having a processor in communication with one or more memory devices, a material library including a substrate material having at least one surface having polymer brushes formed thereon, the polymer brushes presented in a plurality of domains; and one or more functional groups, the functional groups immobilized to the polymer brushes at one or more of the domains, a reading device capable of detecting labels at library domain addresses, the reading device in communication with the processor, an instruction set stored in at least one memory device, the instruction set capable of interacting with the processor, a user controlled input device capable of entering information into the memory device, and an output device in communication with the processor or memory. In one embodiment, the functional groups are polypeptide sequences. In another embodiment, the functional groups are polynucleotide sequences. In still another embodiment, the functional groups are immunoglobulins or antigen binding fragments thereof. In yet another embodiment, the immunoglobulin concentration varies at each domain from about 0.1 fg/mm antibody immobilized per domain surface area to about 100 mg/mm² antibody immobilized per domain surface area. In even another embodiment, the system includes functional groups of human cells, viral cells, or bacterial cells. In one embodiment, the system includes a microfluidic device, wherein the library is contained within a reaction chamber of the microfluidic device. In another embodiment, the processor is in communication with one or more microfluidic ports on the microfluidic device. In one aspect, the system includes a laboratory information management program.

In still another aspect, the invention includes a material library having a substrate material further including a plurality of domains having polymer brushes formed thereon, and a plurality of polypeptide functional groups immobilized to the polymer brushes at one or more of the domains. In another aspect, the invention includes a material library having a substrate material further including a plurality of domains having polymer brushes formed thereon, and a plurality of cells immobilized to the polymer brushes at one or more of the domains. In one embodiment, the cells are human cells. In another embodiment, the cells are human cancer cells. In yet another embodiment, the cells are virally infected cells. In even another embodiment, the cells are viral cells. In still another embodiment, the cells are bacterial cells, for example, pathogenic bacterial strains. In another aspect, the invention includes a material library having a substrate material further including a plurality of domains having polymer brushes formed thereon, and one or more types of immunoglobulin molecules immobilized to the polymer brushes at one or more of the domains. In another aspect, the invention is a material library having a substrate material further including a plurality of domains having polymer brushes formed thereon, and polypeptide functional groups immobilized to the polymer brushes at one or more of the domains, the polypeptide functional groups capable of interacting with one or more targets. In one embodiment, the targets are protein targets, for example from cellular lysates. In yet another embodiment, the cellular lysate includes the soluble fraction derived from pulse labeled cells. In still another embodiment, the cells are human cells. In another embodiment, the polypeptide functional groups include random peptides from about 5 mer to about 50 mer in length. In one embodiment, the interaction of targets with functional groups at one or more domains is detected. In yet another embodiment, detection of interactions includes detection of one or more markers for human disease.

Other features and advantages of the invention will be apparent from following detailed description, and from the claims.

### BRIEF DESCRIPTION OF THE DRAWINGS

FIG. 1 illustrates a schematic for preparing materials having cation and anion exchange functional groups.

FIG. 2 shows an apparatus for introducing cation and anion exchange functional groups to opposing surfaces of an irradiated high density polyethylene (HDPE) film to create graft-polymerized bipolar materials.

FIG. 3 illustrates electrodialysis using graft-polymerized bipolar materials (BP), anion exchange materials (A) and cation exchange materials (C).

FIG. 4 illustrates the relationship between the degree of co-grafting as measured by changes in material thickness as a function of co-grafting time for (a) SSS/HEMA (sodium styrene sulfonate/hydroxyethyl methacrylate) cation exchange materials and (b) VBTAC/HEMA (vinyl benzyl trimethyl ammonium chloride/hydroxyethyl methacrylate) anion exchange materials.

FIG. 5 shows the increase of (a) sulfonic acid group and hydroxyl group densities in the cation-exchange material as well as the increase of (b) quaternary ammonium salt group and hydroxyl group densities as a function of degree of co-grafting.

FIG. 6 illustrates the distribution profiles of sulfonic acid functional groups and the quaternary ammonium functional groups as a measure of the sulfur and the chlorine, respectively, across the thickness of both materials as determined by X-ray microanalysis (XMA).

FIG. 7 illustrates the relationship between functional group density as measured by titration of the salt-splitting capacity and the surface area ratio of X-ray intensity distribution of the (a) sulfur and (b) chlorine functional groups, as measured by XMA.

FIG. 8 shows the increase of the degree of co-grafting and the thickness of the prepared bipolar material as a function of reaction time (a) and the increase in functional group density also as a function of DG (degree of grafting) (b).

FIG. 9 illustrates the functional group density profiles across the thickness of the membranes where DG=16% and DG=86%.

FIG. 10 illustrates the voltage-current characteristic of the co-grafted-type bipolar material.

FIG. 11 shows the time course for electrodialysis using the co-grafted-type bipolar materials having DG=16% (a) and DG=86% (b) as measured by the concentration changes of NaCl (salt chamber), HCl (acid chamber) and NaOH (alkali chamber).

FIG. 12 illustrates the decrease in the voltage and the current during the electrodialysis as a function of operation time using the co-grafted-type bipolar materials having DG=16% and DG=86%.

FIG. 13 shows the electrodialysis efficiency for the co-grafted-type bipolar material.

FIG. 14 illustrates the chemical structure of the grafted-type GMA-DEA-BC fiber having a material of polyethylene (PE) polymer brushes comprising glycidyl methacrylate (GMA) polymers, and functional groups of diethylamine (DEA) quaternized with benzyl chloride (BC).

FIG. 15 shows the conversion of quaternization of the grafted-type GMA-DEA-BC material as a function of reaction time.

FIG. 16 illustrates XMA profiles of chloride ion adsorbed on the grafted-type GMA-DEA-BC materials as a function of conversion by BC.

FIG. 17 shows adsorption of Staphylococcus aureus cells using three grafted-type GMA-DEA-BC materials with different degrees of quaternization.

FIG. 18 shows the relationship between the adsorption rate constant (k) describing the binding of Staphylococcus aureus cells and the functional-group-density of the grafted-type GMA-DEA-BC material.

FIG. 19 illustrates the changes in CFU/mL (colony forming units) and pH of the flow through solution following contact of the Staphylococcus aureus cells with the grafted-type GMA-DEA-BC material, as a function of contact time.

FIG. 20 illustrates a material library comprising functionalized domains fabricated in an array or matrix format, wherein the domains shown vary in terms of polymer brush length and functional group density.

FIG. 21 illustrates the material library of FIG. 20, wherein a target compound is introduced to one or more domains on the library by a microfluidic device capable of varying the composition of an input solution containing the target, and where binding of a target to one or more domains indicates the optimum binding conditions, i.e., the brush length and functional group density, for isolation of the compound contained in the particular input solution.

### DETAILED DESCRIPTION OF THE INVENTION

Definitions

Unless otherwise defined, all technical and scientific terms used herein have the same meaning as commonly understood by one of ordinary skill in the art of which this invention belongs. However, the following terms have the meanings specified below.

As used herein, the term “material” refers to a substrate providing one or more surfaces, where at least one surface is capable of forming grafted polymer brushes, or to which polymer brushes can be otherwise affixed. Thus a material may include two or more different materials. There is also a wide variety of shapes, into which the material may be fabricated or otherwise formed, depending on the particular application for which the material will be used. For example, the material may be substantially rigid. This is appropriate in applications, for example, where the material is formed into e.g., a vial, a pipet tip, a cell culture or ELISA dish, slide or other type of substrate for forming an array or matrix of materials. The material may be substantially flexible along one or more planes, for example formed into a fiber or membrane. The material may be in the form of a powder, a particle preparation or a microparticle suspension. The material may be substantially elongated and flexible, and may define a lumen, for example, fabricated into tubing or pipet tips. A wide variety of materials are appropriate for the materials and methods of making the same which are disclosed herein, and are also described in U.S. Pat. Nos. 6,009,739, 5,783,608, 5,743,940,5,738,775, 5,648,400, 5,641,482, 5,506,188, 5,425,866, 5,364,638, 5,344,560, 5,308,467, 5,075,342, 5,071,880, 5,064,866, 4,980,335, 4,897,433, 4,622,366, 4,539,277, 4,407,846, 4,379,200, 4,376,794, 4,288,467, 4,287,272, 4,283,442, 4,273,840, 4,137,137 and 4,129,617, each incorporated herein by reference.

As used herein, the term “brush” or “polymer brush” refers to a polymeric side chain that is formed from a polymerization substrate having a radical-polymerizable terminal group, wherein the polymerizable substrate is the material itself, or another polymerizable material that can be engrafted to, or otherwise affixed to the material. The side chain can be any reactive monomer (or polymer), but an easily functionalizable reactive polyvinyl polymer is currently preferred, for example such as polyglycidyl methacrylate (GMA) or polyhydroxyethyl methacrylate (HEMA), which has one reactive epoxide group per repeat. Polymer brushes are formed by radical polymerization as described below. A brush has an elongated shape of a particular size in one direction related to the degree of polymerization in a first direction, its “length”, and a cross sectional diameter or thickness related to the degree of polymerization in a second direction perpendicular to the first direction, its “width”. The brushes can assume a coiled or compacted morphology or an extended morphology. The width of a brush can vary along its length. In addition, the polymerization reaction can be controlled to create branch-like polymer brush structures, as well as increasing or decreasing brush density, i.e., number of brushers per surface area or per weight of material, as described below. The length, width, branching, and overall morphology of the polymer brushes in the present invention can be varied according to the desired end use or purpose as described herein and by methods known in the art.

As used herein the term “reactive monomer” refers to a compound that is capable of participating in a radical induced grafting reaction. The reactive monomer can be any material capable of forming polymers as described above and herein, for example but not limited to glycidyl methacrylate (GMA), or ethylene. The material and reactive monomer may be of the same compound, for example, a polyethylene material may utilize ethelyene monomers or polymers in the grafting reaction. A wide variety of reactive monomers are appropriate for the membrane compositions and methods disclosed herein, and are described below and in U.S. Pat. Nos. 6,009,739, 5,783,608, 5,743,940, 5,738,775, 5,648,400, 5,641,482, 5,506,188, 5,425,866, 5,364,638, 5,344,560, 5,308,467, 5,075,342, 5,071,880, 5,064,866, 4,980,335, 4,897,433, 4,622,366, 4,539,277, 4,407,846, 4,379,200, 4,376,794, 4,288,467, 4,287,272, 4,283,442, 4,273,840, 4,137,137 and 4,129,617, each incorporated herein by reference.

As used herein the term “degree of polymerization” refers to the extent of radical induced polymerization of a polymerizable substrate having a radical-polymerizable terminal group, with one or more types of a reactive monomer, wherein the polymerization reaction forms a polymer brush. The degree of polymerization is thus determinative of the overall brush surface characteristics. The polymeric side chains can, for example, be a monomer, an oligomer, or have an average length between about 10 nm and about 2000 nm corresponding to anywhere from about several hundred to tens of thousands of monomer units or longer, for example about 5000 nm or more. The degree of polymerization depends on, e.g., the crystalinity of the polymerizable substrate, the degree of radicalization, the length of time the reaction is allowed to progress, and on the physical properties of the polymerizable substrate, i.e., its strength or rigidity (see, Lee, et al., (1999) Chem, Mater., 11, 3091-3095, incorporated herein by reference).

As used herein the term “degree of grafting” or “DG” refers to the brush density, i.e., the number of the side chains brushes per unit surface area of material. Anywhere from about 1.0×10⁸ to about 1.0×10³⁰ of the side chains brushes can be present per square meter of surface area or per weight of material, for example, from about 1.0×10⁶ to about 1.0×10²⁰ of the side chains brushes represents a degree of grafting between about 10% and about 500%. The degree of grafting is essentially a ratio describing the initial weight of a material and the additional weight of the polymer brush structures (see, Lee, et al., (1999).

As used herein a “functional group” refers to a compound having a particular chemical property, biological activity or affinity for a ligand, or a particular structure. A functional group is immobilized, bound, entrapped, cross-linked or otherwise substantially affixed to the polymer brushes grafted to the material. A wide variety of functional groups are suitable for the present invention, imparting such functionality to the brushes. Combinations of functional groups are preferred where multi-functionalized materials are desirable. A functional group can be any molecule or complex of molecules which has the ability to bind a target and immobilize it to the polymer brush. Preferably, the functional group binds its target in a substantially specific manner. Hence, the functional group may optionally be a target whose natural function in a cell is to specifically bind another protein, such as an antibody or a receptor. Alternatively, the functional group may instead be a partially or wholly synthetic or recombinant polypeptide which specifically binds a target. Alternatively, the functional group may be a polypeptide which has been selected in vitro from a mutagenized, randomized, or completely random and synthetic library by its binding affinity to a specific target. The selection method used may optionally have been a display method such as ribosome display or phage display. Alternatively, the functional group obtained via in vitro selection may be a DNA or RNA aptamer which specifically binds a protein target (for example: Potyrailo et al., Anal. Chem., 70:3419-25, 1998; Cohen, et al., Proc. Natl. Acad. Sci. USA, 95:14272-7, 1998; Fukuda, et al., Nucleic Acids Symp. Ser., (37):237-8, 1997, each incorporated by reference). Alternatively, the in vitro selected functional group may be a polypeptide (Roberts and Szostak, Proc. Natl. Acad. Sci. USA, 94:12297-302, 1997, incorporated by reference). In an alternative embodiment, the functional group may be a small molecule which has been selected from a combinatorial chemistry library or is isolated from an organism. Functional groups may also be selected on their ability to bind a target and catalyze a biological reaction. Suitable functional groups include, for example and without limitation, anionically dissociating groups (e.g., primary, secondary, tertiary, or quaternary amines), cationically dissociating groups (e.g., acid groups) with or without coexisting hydrophilic or hydrophobic groups (nonionic groups such as, GMA or other hydrophobic reactive groups), polypeptides, polynucleotides, proteins or active domains thereof, ions, epitopes and affinity tags, nucleic acids, ribonucleic acids, polypeptides, glycopolypeptides, mucopolysaccharides, lipoproteins, lipopolysaccharides, carbohydrates, enzymes or co-enzymes, hormones, chemokines, lymphokines, antibodies, ribozymes, aptamers, siRNA's, IFN-alpha, IFN-gamma, SpA, SpG, immunoglobulins including monoclonal and polyclonal preparations, TNF-alpha, TNF-beta, v-Ras, c-Ras, reverse transcriptase, G-coupled protein receptors (GPCR's), FcRn, FcγR's, FcεR's, nicotinicoid receptors (nicotinic receptor, GABA_{A} and GABA_{C} receptors, glycine receptors, 5-HT₃ receptors and some glutamate activated anionic channel receptors), ATP-gated channels (also referred to as the P2X purinoceptors), glutamate activated cationic channels (NMDA receptors, AMPA receptors, Kainate receptors, etc.), hemagglutinin (HA), receptor-tyrosine kinases (RTK's) such as EGF, PDGF, NGF and insulin receptor tyrosine kinases, SH2-domain proteins, PLC-γ, c-Ras-associated GTPase activating protein (RasGAP), phosphatidylinositol-3-kinase (PI-3K) and protein phosphatase 1C (PTP1C), as well as intracellular protein tyrosine kinases (PTK's), such as the Src family of tyrosine kinases, protein-tyrosine phosphatases, such as receptor tyrosine phosphatase rho, protein tyrosine phosphatase receptor J, receptor-type tyrosine phosphatase D30, protein tyrosine phosphatase receptor type C polypeptide associated protein, protein tyrosine phosphatase receptor-type T, receptor tyrosine phosphatase gamma, leukocyte-associated Ig-like receptor 1D isoform, LAIR-1D, LAIR-1C, MAP kinases, neuraminidase (NA), proteases, polymerases, serine/threonine kinases, second messengers, antigenic or tumorigenic markers, transcription factors, and other such important metabolic building blocks or regulators, and fluorescent labeled polypeptides or fluorescent polypeptides like GFP, growth factor receptors, hormone receptors, neurotransmitter receptors, catecholamine receptors, amino acid derivative receptors, cytokine receptors, extracellular matrix receptors, lectins, serpins, hydrolases, steroid hormone receptors, transcription factors, heat-shock transcription factors, DNA-binding proteins, zinc-finger proteins, leucine-zipper proteins, homeodomain proteins, intracellular signal transduction modulators and effectors, apoptosis-related factors, DNA synthesis factors, DNA repair factors, DNA recombination factors, cell-surface antigens, disease markers, hepatitis C virus (HCV) proteases or HIV proteases. Selection and use of functional groups will depend on the application desired, as illustrated herein.

The term “anionically dissociating functional groups” as used herein means those ion-exchange groups whose counter ion is an anion. Anionically dissociating groups have the ability catalyze chemical reactions and to absorb and/or immobilize target compounds or other functional groups and are capable of entering into neutralizing reactions with acidic substances such as hydrogen sulfide or mercaptans, allowing for a wide range of uses with effective removal of the acidic substances.

The term “cationically dissociating functional groups” as used herein means those ion-exchange groups whose counter ion is a cation. A typical cationically dissociating group is an acid group. Cationically dissociating groups have the ability to catalyze chemical reactions and adsorb and/or immobilize target compounds or other functional groups and are capable of releasing a proton (hydrogen ion) to enter into neutralizing reaction with basic substances, say, ammonia or amines. As a result, these groups provide a wide range of uses with basic substances.

The term “hydrophilic functional groups” as used herein refers to groups that have an affinity for water but do not undergo significant ionic dissociation upon contact with water. Hydrophilic groups have the ability to catalyze chemical reactions and adsorb and/or immobilize target compounds or other functional groups, by providing a hydration shell, or by providing a reactive surface. An example of such group, without limitation, is a hydroxyl group.

The term “hydrophobic functional groups” as used herein refers to groups that do not have an affinity for water. Hydrophobic groups have the ability to catalyze chemical reactions and adsorb and/or immobilize target compounds or other functional groups, by excluding water, or by providing a surface for hydrophobic interactions, or by providing a reactive surface. An example of such group, without limitation, is a nonionic group, an ester group, a succinimide group or an epoxy group.

### DETAILED DESCRIPTION OF THE INVENTION

The present invention provides for compositions and methods of immobilizing functional groups to polymer brushes grafted to one or more materials. Immobilization methods include entrapment, gelification, physical retention or adsorption, ionic binding, covalent binding or cross-linking (see, Biotechnol. Bioeng., 22:735-756, 1980; Chem. Eng. Prog., 86:81-89, 1990; J. Am. Chem. Soc., 117:2732-2737, 1995; Enzyme Microb. Technol., 14:426-446, 1997; Trends Biotechnol., 13:468-473, 1997; Nat. Biotechnol., 15:789-793, 1997, each incorporated herein by reference). The immobilization method and the amount and kind of the functional groups used both determine the activity of the composition of the present invention. The resulting activity of the immobilized functional group can often further reduced by mass-transfer effects (see, Methods Enzymol., 44:397-413, 1976; J. Am. Chem. Soc., 114:7314-7316, 1992; Trends Biotechnol., 14:223-229, 1996; Angew. Chem., 109:746-748, 1997, each incorporated herein by reference). The activity following immobilization can be further reduced as a result of the diminished availability of the functional groups, i.e., due to steric hindrance, entrapment within brushes, pores or other structures on the material substrate, or by slow diffusion of the functional groups. Such limitations lead to lowered efficiency. It is an objective of the present invention to provide materials having a high capacity for functional groups immobilized thereto,

The invention is usable with a wide variety of materials, i.e., all polymeric plastics, such as, for example, polyurethanes, polyamides, polyesters, polyethers, polyether-block amides, polystyrene, polyvinyl chloride, polycarbonates, polyorganosiloxanes, polyolefins, polysulfones, polyisoprene, polychloroprene, polytetrafluoroethylene (PTFE), corresponding copolymers and blends, as well as natural and synthetic rubbers, metal, glass or wooden bodies. The compositions have multifunctional properties and can be used to separate, remove, purify, synthesize, concentrate and immobilize compounds, and are particularly suited to the harsh operating environments, i.e., extreme temperatures and pressures, chemical concentrations, electrical charges, etc., from commercial processes.

In general, the desired target compound is in a sample solution, which can be passed directly through the compositions, as in a filtration membrane, tube, pipet tip or a chromatography matrix. Liquids containing cells or other large insoluble particles may require pre-treatment to separate the larger particles from the smaller soluble ones. However, the polymer brush sizes and brush density provide a degree of physical filtration, and the compositions can be woven or otherwise fabricated into filtration devices if appropriate. While an aqueous sample solution is often described, one skilled in the art will realize that gaseous samples may be employed. Examples of filter elements for adsorbing gaseous components of a gas stream are described in, for example, U.S. Patent Application 20020002904 A1, to Gentilcore, et al., published Jan. 10, 2002, herein incorporated by reference. In addition, a membrane or fiber is often described, but the compositions of the invention illustrated below can comprise other forms as described herein. Thus the following is illustrative and are not meant to be limiting examples of the present invention.

Materials Useful in the Present Invention

In general, the material of the present invention is not limited to any particular type, and any substrate that permits grafting or affixation of the polymer brush is an appropriate material. Treatment of a material surface is acceptable if the original material is not itself sufficient for the polymerization reaction. In such cases, the surface treatment according to the invention can be, for example, a coating formed from a polymeric material. Materials useful in the present invention are widely available, for example polyolefins (low density or high density) including polyethylene and polypropylene, cellulose (see, Radiat. Phys. Chem. 1990, 36:581; J. Membr. Sci. 1993, 85:71), poly(isobutylene oxide) (see, Radiat. Phys. Chem. 1987, 30:151), ethylene-tetrafluoroethylene copolymer (see, J. Electrochem. Soc. 1996, 143: 2795) ethylene-propylene-diene terpolymer (see, Radiat. Phys. Chem. 1991, 37: 83) ethylene-propylene rubber (see, Nippon Gensiryoku Gakkaishi, 1977, 19:340) chlorosulfonated polyethylene (see, Radiat. Phys. Chem. 1991, 37:83) polytetrafluoroethylene (PTFE) (see, React. Polym. 1993, 21:187; Radiat. Phys. Chem. 1989, 33:539) tetrafluroethylene-hexafluoropropylene copolymer (see, Radiat. Phys. Chem. 1988, 32:193) poly(vinyl chloride) (see, Radiat. Phys. Chem. 1978, 11:327) silicone rubber (see, Radiat. Phys. Chem. 1988, 32: 605) polyurethanes (see, Radiat. Phys. Chem. 1981, 18: 323) polyesters (see, Radiat. Phys. Chem. 1988, 31: 579) butadiene-styrene copolymer (see, Radiat. Phys. Chem. 1990, 35: 132) natural and nitrile rubbers (see, Radiat. Phys. Chem. 1989, 33: 87) cellulose acetate and propionate (see, Radiat. Phys. Chem. 1990, 36: 581) starch and cotton fabric (see, Zhurn, Vsesoyuz. Khim. Ob-va im. D. I. Mendeleeva. 1981, 26:401) polyester-cellulose fabric (see, Radiat. Phys. Chem. 1981, 18:253) natural leather (see, Radiat. Phys. Chem. 1980, 16:411) and medical gauze (see, Zhum. Vsesoyuz. Khim. Ob-va im. D. I. Mendeleeva. 1981, 26:401) hydrophilic polyurethanes, polyureas, olefins, acrylics, as well as other hydrophilic components. Particular materials include polyethylene glycol, polyethylene glycol or polypropylene glycol copolymers and other poloxamers, heterocyclic monomers (see, Applied Radiation Chemistry: Radiation Processing, Robert J. Woods and Alexei K. Pikaev, John Wiley & Sons, Inc., 1994 (ISBN 0-471-54452-3)), poly(ethylene glycol) methacrylate or dimethacrylate (see, J. Appl. Polym. Sci., 1996, 61:2373-2382), polyamine (such as polyethyleneimine), poly(ethylene oxide), and styrene. These coatings preferably are covalently bonded to the surface which is being treated. Many methods for forming the coating exist, and include the steps of adsorbing the polymeric material to the surface, and then covalently attaching the polymeric material to the surface by exposure to UV radiation, RF energy, heat, X-ray radiation, gamma radiation, electron beams, chemical initiated polymerization or the like.

A material provides a plurality of surfaces, and may be itself a polymerizable substrate having a radical-polymerizable terminal group, for example, celluloses, polyolefins, polyacrylonitriles, polyesters such as PET and PBT, polyamides such as nylon 6 and nylon 66, as well as combinations of these. An appropriate material may not itself be polymerizable, but is suitable for the present invention provided polymer brushes can be grafted, affixed, or otherwise adhered to the non-polymerizable material.

A carbohydrate polymer, such as cellulose or lignin, or a similar material, can be used as the material. An example of a composition and method of a grafted carbohydrate polymer having pendant 3-amino-2-hydroxy propyl groups grafted thereon, for use as a retention aid and strengthening additive in paper manufacture is described in U.S. Patent Application 20020026992 A1, to Antal, et al., published Mar. 7, 2002, incorporated herein by reference. The method of radiation induced grafting to cellulose is described in, Yamagishi et al., (1993) J. Membr. Sci., 85, 71-80, incorporated herein by reference.

When the carbohydrate polymer is a component of wood pulp the resulting chemically modified wood pulp may be employed in conjunction with unmodified wood pulp to incorporate therein the retention and strengthening characteristics. Typical sources of the carbohydrates, specifically celluloses that can be used as the material include wood celluloses such as paper pulp and wood chips. In addition to these celluloses, leaf fiber cellulose, stem fiber cellulose and seed tomentous or pubescent fiber cellulose can also be used. Examples of such celluloses include bast fibers (e.g., hemp, flax, ramie and Manila hemp) and cotton. If desired, rice straw, coffee bean husk, spent tea leaves, soy pulp and other waste can be recycled for use as cellulose. Such waste is very convenient to use as a material because it does not require any special preliminary treatments. One such source for cellulose for use in the present invention is paper pulp.

Metallic materials can be grafted with biologically active compounds, for example surface-modified medical metallic materials having a gold or silver thin layer plated onto a base metal, as described in U.S. Patent Application 20010037144, A1 to Kim, et al., published Nov. 1, 2001 and incorporated herein by reference.

Animal tissues such as fiber, hair, and leather can be used as the material. One skilled in the art would be able to determine if an animal product provided the desired properties for use as a material. For example, where it is desired that the invention be used in a mechanical filtration, fibers, for example, can be woven or otherwise fabricated into among other forms; membrane compositions or sheets. Examples of fibers or animal hairs that can be used as materials include wool, camel hair, alpaca, cashmere, mohair, goat hair, rabbit hair, and silk. Examples of natural leather that can be used as materials include cowskin, goatskin, and the skin or hide of reptiles. Examples of synthetic leather that can be used as materials include CORFAM® (DuPont), CLARINO® (Kuraray), and ECSAINE® (Toray).

Polyolefins can also be used as materials (see, Applied Radiation Chemistry: Radiation Processing, Robert J. Woods and Alexei K. Pikaev, John Wiley & Sons, Inc., 1994 (ISBN 0-471-54452-3), Introduction to Radiation Chemistry 3^{rd} Edition, J. W. T. Spinks and R. J. Woods, John Wiley & Sons, Inc., 1990 (ISBN 0-471-61403-3), Radiation Chemistry of Polymeric Systems, A. Chapiro, Interscience, New York, 1962, Atomic Radiation and Polymers, A Charlesby, Pergamon Press, 1960, Radiat. Phys. Chem. 1991, 37:175-192, and Prog. Polym. Sci. 2000, 25:371-401 (all incorporated herein by reference in their entirety). Polyolefins can be fabricated into many shapes and forms. They are capable of being molded, thermoformed, poured, extruded and otherwise shaped by processes well known in the art, such as the formation of fibers or filaments by conventional melt spinning processes. In addition, polyolefin compounds are useful in among other industries, the biotechnology industry, largely because polyolefin products are resistant to chemical degradation from common laboratory reagents, are durable and can be reused, and are chemically inert, and are inexpensive and often disposable. Polyolefin compounds are currently preferred materials as they demonstrate these properties and additionally provide a polymerizable substrate having a radical-polymerizable terminal group. Olefin monomers and polymers are well suited to the grafting techniques of the invention both as materials and additionally as reactive monomers. Examples of polyolefins include, for example, polyethylene and polypropylene. If desired, these materials can be modified, for example by incorporating halogens into the polymer, such as chlorine, fluorine, or bromine, for example the halogenated polyolefin, polytetraflurorethylene. Other modifications such as incorporation of hydroxyl groups into the polymer are also appropriate. Polyolefinic polymers having weight-averaged molecular weights in the range of from 20,000 to 750,000 daltons are suitable for the present invention. One skilled in the art would know which molecular weights are appropriate for the particular purpose. For example, a polyolefin having a molecular weight from about 50,000 daltons to about 500,000 daltons is suitable to use in the production of fiber or filament, used for example, in a membrane comprising polyolefin filaments or fibers (see, above) further comprising brushes having combinations of functional groups affixed thereto. When the molecular weight of a polyolefin is greater than about 500,000 daltons, the fluidity of the resultant polyolefin is low, and it is difficult to form the polyolefin into such a filament by conventional melt spinning processes. However, the structural rigidity of a polyolefin greater than about 500,000 daltons is suitable, for example, in high density applications such as containers, freezing vials for cells, and the like. By contrast, when the molecular weight of a polyolefin is lower than about 50,000 daltons, the strength and rigidity of the polymer is lessened and a filament obtained therefrom does not have a sufficient tensile strength. However the structural rigidity of a polyolefin when the molecular weight of a polyolefin is lower than about 50,000 daltons is suitable, for example, in a powder or microcrystaline composition. An example of a polymerized grafted and crosslinkable thermoplastic polyolefin powder composition in the form of a powder intended for the production of flexible coatings by its free flow over a hot mold is described in U.S. Patent Application 20020019487 A1, to Valligy, et al., published Feb. 14, 2002, hereby incorporated by reference. Another polymerized grafted and crosslinkable thermoplastic polyolefin powder composition is described in EP0409992, incorporated by reference, is directed to a process for the preparation of particles of crosslinkable thermoplastic polyolefin polymers according to which said particles are brought into contact, in the solid state, with the crosslinking agent, in particular by means of a mineral oil.

The shape of the material is not limited in any particular way, and various shapes can be employed as selected from among fibers, films, flakes, powders, sheets, mats and spheres. The material of the present invention has the function of serving as a structural member that supports the polymer brushes. From the viewpoint of maximizing the area of adsorption and/or immobilization and enhancing the efficiency of adsorption and/or immobilization, the use of fibrous materials is advantageous. Grafted fibers in porous hollow fiber membrane configurations or woven or otherwise fabricated into sheets provide two examples of a substantially enhanced brush surface area. Materials with lumenal inner surfaces can also be adapted to enhance brush surface area by manufacturing the material such that the lumenal surface contains irregularities or projections. An analogous illustration of this embodiment may be found in physiology, using the small intestine as an example: the mucosal surface contains villiar projections (i.e., the surface irregularities) and upon each villus are thousands of microvillar structures (i.e., the polymer brushes).

Woven fiber sizes appropriate for the present invention range from about 10 nm to about 100,000 nm. It is particularly advantageous to use woven fibrous materials having fiber diameters of from about 1000 nm to about 50,000 nm. One of the reasons why fibrous materials are advantageous is that they can be easily worked or woven into a desired shape, i.e., a fabric, and assembled in a device. Further, fibrous materials generally have no potential to release fine particles or dust into the atmosphere and, hence, they can be used in semiconductors and other areas of precision machining. If fibrous materials are to be used, they can be staple fibers or filaments. Such fibers can be processed into woven or nonwoven fabrics. If the membrane of the present invention employs a fibrous substrate, it can be used in admixture with other fibrous materials. Combinations of fibers thereby comprising different functional groups can be fabricated, thus providing for multifunctional properties in a single membrane composition.

Fibers can also be porous hollow fibers manufactured as nonwoven substrates. Examples of commercially available porous hollow fibers are those manufactured by Asahi Chemical Industry, Corp., described herein. These can have a broad range of porosity and be fabricated into, for example, filtration devices. Furthermore, a combination of porosity and fiber composition thereby provides physical and molecular immobilization, filtration or concentration. If fibrous materials are to be used in a spherical form, their diameter is advantageously adjusted to lie between about 2 and 20 mm, simply from the viewpoint of ease of handling. The porosity of the material of the present invention has an average pore diameter of about 0.1 nm to about 50,000 nm, and preferably about 1 nm to 5000 nm, and more preferably 10 to 1000 nm from the standpoint of the desired functional activity and permeability of the material. One skilled in the art could determine the optimal composition and porosity for a given application. When the average pore diameter is too small, the permeability of the membrane composition is decreased. When the average pore diameter is too large, desired substances would are not well adsorbed on the brush surface of the porous material. Instead, the subject sample would pass through the pores of the porous material without contacting the brush surface and functional groups, so that the activity of the desired functional group cannot be attained. The porosity of the porous material of the present invention is preferably in the range of from 20 to 90%, more preferably 50 to 90%. The degree of porosity depends e.g., on the physical properties of the material used. Measurement of porosity and pore size etc. of a material is generally well known in the art, for example, the bubble point method, mercury pressure method, Scanning Electron Microscopy (SEM) or Tunneling Electron Microscopy (TEM) or the nitrogen adsorption method (see, ASTM F316, 1970; Pharmaceutical Tech., 1978, 2:65-75; Filtration in the Pharmaceutical Industry, Marcel Dekker, 1987, incorporated herein by reference).

Agents for Generating Radicals

The agent generating radicals which are capable of creating radical sites is an organic peroxide or a perester such as, for example, tert-butylperoxy 3,5,5-trimethylhexanoat-e,2,5-dimethyl-2,5-di(benzoylperoxy)hexane, tert-butyl-peroxy 2-ethylhexyl carbonate, tert-butylperoxy acetate, tert-amylperoxy benzoate, tert-butylperoxy benzoate, 2,2-di(tert-butylperoxy)butane, n-butyl 4,4-di(tert-butyl-peroxy)valerate, ethyl 3,3-di(tert-butylperoxy)-butyrate, dicumyl peroxide, tert-butyl cumyl peroxide, di-tert-amyl peroxide, di(2-tert-butylperoxyisopropyl)benzene, 2,5-dimethyl-2,6-di(ter-t-butylperoxy)hexane, di-tert-butyl peroxide, 2,5-dimethyl-2,5-di-tert-but-ylperoxy-3-hexyne, 3,3,6,6,9,9-hexamethyl-1,2,4,5-tetraoxacyclononane, tert-butyl hydroperoxide, 3,4-dimethyl-3,4-diphenylhexane, 2,3-dimethyl-2,3-diphenylbutane and tert-butyl perbenzoate and azo compounds, for example azobisisobutyronitrile and dimethyl azodiisobutyrate; the said agent is preferably chosen within the group consisting of dicumyl peroxide, tert-butyl cumyl peroxide, di-tert-amyl peroxide, di-tert-butyl peroxide and 2,5-dimethyl-2,5-di(tertbutylperoxy)-3-hexyne.

Radiation Induced Graft Polymerization

Graft polymerization can be carried out, for example, by polymerization in the presence of a chemical or inducible polymerization initiator, thermal polymerization, irradiation-induced polymerization using ionizing radiation (e.g., alpha rays, beta rays, gamma rays, accelerated electron rays. X-rays, or ultraviolet rays). Polymerization induced by gamma rays or accelerated electron rays provides a convenient graft polymerization method.

Several methods of graft polymerization of a reactive monomer to a material exist. The material can be a formed article or can be manufactured into a product or device at a later time. Liquid phase polymerization, in which a formed article is directly reacted with a liquid reactive monomer, and gaseous or vapor phase polymerization, in which a formed article is brought into contact with vapor or gas of a reactive monomer, are two polymerization methods that are useful in the present invention according to the end use or purpose. Vapor phase grafting is described in J. Membr. Sci. 1993, 85:71-80, Chem. Mater. 1991, 3:987-989, Chem. Mater. 1990, 2:705-708, and AIChE J. 1996, 42:1095-1100, all of which are herein incorporated by reference.

Graft polymerization of the reactive monomer to the material is performed. Grafting proceeds in three different ways: (a) pre-irradiation; (b) peroxidation and (c) mutual irradiation technique. In the pre-irradiation technique, the first polymer backbone is irradiated in vacuum or in the presence of an inert gas to form radicals. The irradiated polymer substrate is then treated with the monomer, which is either liquid or vapor or as a solution in a suitable solvent. However, in the peroxidation grafting method, the trunk polymer is subjected to high-energy radiation in the presence of air or oxygen. The result is the formation of hydroperoxides or diperoxides depending on the nature of the polymeric backbone and the irradiation conditions. The peroxy products, which are stable, are then treated with the monomer at higher temperature, whence the peroxides undergo decomposition to radicals, which then initiate grafting. The advantage of this technique is that the intermediate peroxy products can be stored for long periods before performing the grafting step. On the other hand, with the mutual irradiation technique the polymer and the monomers are irradiated simultaneously to form the radicals and thus addition takes place. Since the monomers are not exposed to radiation in the preirradiation technique, the obvious advantage of that method is that it is relatively free from the problem of homopolymer formation which occurs with the simultaneous technique. However, the decided disadvantage of the pre-irradiation technique is the scission of the base polymer due to its direct irradiation, which brings forth predominantly the formation of block copolymers rather than graft copolymers.

The material substrate surfaces activated in this way are coated in a solution including reactive monomers, for example, tert-butylaminoethyl methacrylate, by known methods, such as by dipping, spraying or brushing. Suitable solvents have proved to be water and water/ethanol mixtures, although other solvents can also be used if they have a sufficient dissolving power for tert-butylaminoethyl methacrylate, and wet the material substrate surfaces thoroughly. Solutions having reactive monomer contents of 0.1% to 10% by weight, for example about 5% by weight, have proved suitable in practice and in general give continuous coatings which cover the substrate surface and have coating thicknesses which can be more than 0.1 μm in one pass. Two, three, or more different reactive monomers can be cografted to the material, see, Chem. Mater. 1999, 11:1986-1989, J. Membr. Sci. 1993, 81:295-305, J. Electrochem. Soc. 1995, 142:3659-3663, and React. Polym. 1993, 21:187-191, all incorporated herein by reference.

A reactive monomer is any compound that is capable of participating in a radical induced graft polymerization reaction. The reactive monomer thus incorporates in the side chain reaction, and forms polymer brushes. The term monomer is used for simplicity, as side reactions between reactive monomers can create oligomers before these are in turn involved in the polymerization reaction with the material, and oligomers or even polymers are also useful reactive species for the present invention. As described above, monomer side chain brushes can be obtained, comprising multiple functional groups, i.e., three functional groups on a single monomeric brush.

The material and reactive monomer may be the same compound, for example, a polyethylene material may utilize ethylene monomers or polymers in the grafting reaction. Reactive monomers that can be used in the present invention include, for example, vinyl monomers and heterocyclic monomers. Other specific examples of suitable reactive monomers include vinyl monomers containing a glycidyl group, e.g., glycidyl methacrylate, glycidyl acrylate, glycidyl methylitaconate, ethyl glycidyl maleate, and glycidyl vinyl sulfonate; and vinyl monomers containing a cyano group, e.g., acrylonitrile, vinylidene cyanide, crotononitrile, methacrylonitrile, chloroacrylonitrile, 2-cyanoethyl methacrylate, and 2-cyanoethyl acrylate. These have epoxide groups for immobilization of functional groups and vinyl groups, which provide reactive polymerization sites and are thereby useful as reactive monomers. Ring-opening, i.e., the conversion of the epoxy groups into diol groups of the poly-GMA brushes is described in J. Membr. Sci. 1996, 117:33-38 (incorporated by reference).

The reactive monomers are covalently bonded to the material through the polymerization reaction, or are separately formed and affixed or adhered to the material. The reactive monomers form polymer brushes that are thereby grafted to the material. The degree of grafting is determined by the choice of material and reactive monomer, the polymerization method, and the desired length and width of the brushes. In certain cases, the resultant polymer brushes of the invention have bioactive properties themselves, for example, tert-butylaminoethyl methacrylate on a surface of an article or apparatus displays antimicrobial activity.

Measurement of modified or grafted materials can be determined by, for example degree of grafting, assaying thickness or weight, water content, IR method (FTIR-ATR, etc), titration for ion-exchange groups, zeta-potential, Donnan method, atomic force microscopy (AFM), scanning electron microscopy (SEM), determination of contact angle, XPS (X-ray photoelectron spectroscopy), XMA (x-ray microanalysis), and SIMS (secondary ion mass spectrometry).

The grafting copolymerization of the reactive monomer applied to the activated surfaces is also effected by radical induced polymerization initiated by, for example, short wavelength radiation in the visible range or in the long wavelength segment of the UV range of electromagnetic radiation. The radiation of a UV-Excimer of wavelengths 250 to 500 nm, preferably 290 to 320 nm, for example, is particularly suitable. Mercury vapor lamps are also suitable here if they emit considerable amounts of radiation in the ranges mentioned. The exposure times generally range from 10 seconds to 30 minutes, preferably 2 to 15 minutes. A suitable source of radiation is, for example, a UV-Excimer apparatus HERAEUS Noblelight, Hanau, Germany. However, mercury vapor lamps are also suitable for activation of the substrate if they emit considerable proportions of radiation in the ranges mentioned. The exposure time generally ranges from 0.1 second to 20 minutes, preferably 1 second to 10 minutes.

The activation of the reactive monomers and materials with UV radiation can furthermore be carried out with an additional photosensitizer. Suitable such photosensitizers include, for example, benzophenone, as such are applied to the surface of the substrate and irradiated. In this context, irradiation can be conducted with a mercury vapor lamp using exposure times of 0.1 second to 20 minutes, preferably 1 second to 10 minutes.

According to the invention, the activation can also be achieved by a high frequency or microwave plasma (Hexagon, Technics Plasma, 85551 Kirchheim, Germany) in air or a nitrogen or argon atmosphere. The exposure times generally range from 30 seconds to 30 minutes, preferably 2 to 10 minutes. The energy output of laboratory apparatus is between 100 and 500 W, preferably between 200 and 300 W. For example, a Corona apparatus (SOFTAL, Hamburg, Germany) can furthermore be used for the polymer activation. In this case, the exposure times are, as a rule, 1 to 10 minutes, preferably 1 to 60 seconds.

The flaming of surfaces likewise leads to activation of the reactive monomers and materials. Suitable apparatus, in particular those having a barrier flame front, can be constructed in a simple manner or obtained, for example, from ARCOTEC, 71297 Monsheim, Germany. The apparatus can employ hydrocarbons or hydrogen as the combustible gas. In all cases, harmful overheating qf the materials must be avoided, which is easily achieved by intimate contact with a cooled metal surface on the substrate surface facing away from the flaming side. Activation by flaming is accordingly limited to relatively thin, flat materials. The exposure times generally range from 0.1 second to 1 minute, preferably 0.5 to 2 seconds. The flames without exception are nonluminous and the distances between the substrate surfaces and the outer flame front ranges from 0.2 to 5 cm, preferably 0.5 to 2 cm.

In the case of ionizing radiation initiated polymerization, in addition to the ultraviolet radiation discussed above, electron beams, X-rays, alpha rays, beta rays, gamma rays, etc., can be used. Graft polymerization condition changes with such variables, as the crystalline and amorphous structure of the material polymer, the influence of solvent or gasses, temperature, pH, the hydrophobicity/hydrophilicity of the material, reactive monomers, irradiation dose and intervals of exposure, and the type of radicals generated by irradiation. One skilled in the art would recognize such variables and adjust experimental conditions accordingly, for example activation by electron beams or gamma-rays, from a cobalt-60 source allow short exposure times which generally range from about 0.1 to about 60 seconds and employ dose ranges of about 1 to about 500 kGy. These high energy radiation sources are appropriate for applications where it is desirable to initiate a radical induced polymerization reaction on one or more intraluminal surfaces of a material.

Multiple grafting steps can also be used to create the polymer brushes. Radicals are generated in the material, for example a polymer material is irradiated at an ambient temperature under nitrogen atmosphere to create radicals for polymer grafting. In the currently preferred embodiment, irradiation is performed by using an electron beam accelerator. Graft polymerization of reactive monomers (for example, liquid phase grafting) is performed on the material to allow the formation of polymer brushes. As such, grafted polymer #1 is obtained. The above processes are repeated to obtain grafted polymer #2, grafted polymer #3 and so on. Moreover, the grafting process can be stopped at any step depending on the desired complexity of the brush structure. Different reactive monomers can be used at each grafting step, providing a plurality of brush compositions for immobilizing numerous types of functional groups or bioactive molecules thereto. The process can include immobilization of functional groups followed by additional grafting reactions.

Functionalized Polymer Brushes

The present invention provides materials having grafted polymer brushes, and methods of making the same as. While these polymer brush structures are designed to have specific physical properties themselves, due to, for example, their size, brush density and brush morphology, the invention provides that these polymer brushes may be functionalized, i.e., the brushes have one or more types of functional groups immobilized thereto. Methods of immobilizing functional groups to particular substrates are known in the art, and are applicable for immobilizing the same to the present materials using the teachings provided herein. One or more types of functional groups can be immobilized to the materials, i.e., one, two, three, four, or five or more different types of functional groups may be used, depending on the desired functionality.

Agents for Immobilizing Functional Groups to the Brushes

The polymer brush structures of the present invention include reactive groups on the brush surface, thus permitting the immobilization of functional groups thereto resulting in materials having functional or multifunctional properties. Different methods for immobilization of functional groups to the polymer brushes include, for example, physical adsorption (non-covalent bridges such as ionic and hydrogen bonds, hydrophobic interactions and van der Waals forces), immobilization via reactive groups, aminopropyltriethoxysilane bridges, glutaraldehyde, or bis(sulfosuccinimidyl) suberate activation, or via aldehydye groups, phosphoramidite groups, peptide groups, binding through biotin or avidin, protein A or G, attachment via metal-carrying media, such as chelate-forming iminodiacetate groups, copper ions, nickel ions, fernic or ferrous ions, zinc ions, magnesium ions, manganese ions, cobalt ions or similar charged species including complexes of the same, covalent attachment of oxidized groups, for example to oxidize the carbohydrate moieties in an antibody's Fc region with periodate to form aldehyde groups, which are then chemically bound to hydrazide-activated solid supports such as agarose, silica, acrylic-based copolymers, and cellulose. Methods for immobilization of nucleic acids include, for example, adsorption: (i) electrochemical adsorption: electrostatic attraction between the positively charged solid support and the negatively charged oligonucleotides. (ii) hybridization between electrochemically adsorbed oligonucleotides and its complementary target for sequence specific hybridization, avidin-biotin complexation, covalent attachment: (i) through deoxyguaosine group using carbodiimide method (in other words, carboxylic group (—COOH)), (ii) amino groups (—NH2), phosphoric acid groups. Organic synthesis (or peptide synthesis) can be performed directly on the polymer brushes or on functional groups immobilized thereto (see, U.S. Pat. No. 6,306,975, incorporated by reference). Other coupling chemistries are well known in the art, and by using graft polymerization, one can prepare solid supports having a plurality of functional groups (see, J. Biochem. Biophys. Methods 2001, 49:467-480, Radiat. Phys. Chem. 1987, 30:263-270, Biosens. Bioelectron. 2000, 15:291-303, Analytica Chimica Acta 1997, 346:259-275, Chem. Rev. 2000, 100: 2091-2157, Tetrahedron 1998, 54: 15383-15443, Radiat. Phys. Chem. 1986, 27:265-273, and Solid-Phase Synthesis and Combinatorial Technologies by Pierfausto Seneci, John Wiley & Sons, Inc., 2000, all incorporated herein by reference).

Another method of immobilizing a molecule to the brush surface includes, without limitation, silanes of the formula SiX3-R, wherein X is a methyl group or a halogen atom such as chlorine and R is a functional group which can be a coating material as described herein or a group which is reactive with a coating material. Particular silane-terminated compounds include vinyl silanes, silane-terminated acrylics, silane-terminated polyethylene glycols (PEGs), silane-terminated isocyanates and silane-terminated alcohols. The silanes can be reacted with the surface by various means known to those skilled in the art. For example, dichloro methyl vinyl silane can be reacted with the surface in aqueous ethanol. This strongly binds to the surface via —O—Si bonds or directly with the silicon atom. The vinyl group of the silane can then be reacted with polymeric materials as described herein using appropriate conventional chemistries. For example, a methacrylate-terminated PEG can be reacted with the vinyl group of the silane, resulting in a PEG that is covalently bonded to the surface of the present device.

In addition, spacer molecules may be inserted between the functional group and the polymer brush, as is known in the art, to facilitate binding or improve the activity of the functional group or bioactive molecule. The extended morphology of the brushes can function as spacers, or additional chemical spacers can be used. Branch-like grafted brush structures often provide optimal steric positioning of larger functional groups, or ones having larger cognate targets.

These functional groups impart to the compositions of the invention particular properties. For example, the functional groups can change the effective or active surface area and thereby change the adsorptive capacity. In certain embodiments, they provide for particular brush shapes. In other embodiments they impart a particular strength, chemical resistance, enzymatic property, affinity for a target, or provide other effective functionality to the materials, such as the ability to catalyze a reaction or deliver a compound to cells.

Functional groups that are appropriate for immobilization by the brushes in the compositions of the present invention include, for example, ion exchange functional groups, i.e., anionically dissociating groups and cationically dissociating groups, hydrophilic functional groups, affinity functional groups, enzymatic functional groups, and other such functional groups that have the ability to adsorb and/or immobilize other molecules, or cause a catalytic reaction, alone or in combination with mechanical or physical properties of the polymer brush structures, i.e., adjusting the density and morphology of the brushes to optimize the surface area of a charged group, or to increase the activity of catalytic sites, or sterically optimize the binding sites of the functional groups immobilized thereto, thereby optimizing their association with a particular ligand or target.

One or more kinds of anionically dissociating substances can be immobilized by the polymer brushes. Examples of suitable anionically dissociating groups include quaternary ammonium salts and primary, secondary, and tertiary amino or amido groups. Specific examples include an amino group, a methylamino group, a dimethylamino group, and a diethylamino group. Preferred anionically dissociating groups include the amino group and quaternary ammonium salts. Reactive monomers that have such anionically dissociating groups and that are useful in the present invention include, for example, vinylbenzyltrimethyl ammonium salt, diethylaminoethyl methacrylate, dimethylaminoethyl acrylate, dimethylaminoethyl methacrylate, diethylaminoethyl acrylate, diethylaminomethyl methacrylate, tertiary-butylaminoethyl acrylate, tertiary-butylaminoethyl methacrylate and dimethylaminopropylacrylamide. Also useful in the present invention are reactive monomers that have epoxide groups capable of conversion to anionically dissociating groups. An example of such a reactive monomer is glycidyl methacrylate. An example of an amine capable of converting the epoxide group to an anionically dissociating group is diethylamine.

One or more kinds of cationically dissociating groups can be immobilized by the polymer brushes. Examples of such cationically dissociating groups include, for example, a carboxyl group, a sulfone group, a phosphate group, a sulfoethyl group, a phosphomethyl group, a carbomethyl group. Preferred cationically dissociating groups include a sulfone group and a carboxyl group. Reactive monomers that have such cationically dissociating groups and that are useful include, for example, acrylic acid, methacrylic acid, styrenesulfonic acid and salts thereof, and 2-acrylamido-2-methylpropanesulfonic acid.

One or more kinds of hydrophilic substances can be immobilized by the polymer brushes. Such hydrophilic groups are capable of trapping the water molecules present in air, forming a layer of adsorbed water on the surface of the membrane of the present invention. Such hydrophilic groups will function in water in the same manner as in air. Examples of suitable hydrophilic groups include, for example, a hydroxyl group, a hydroxyalkyl group (where the alkyl group is preferably a lower alkyl group), an amino group and a pyrrolidonyl group. Preferred hydrophilic groups include a hydroxyl group, a hydroxyalkyl group and a pyrrolidonyl group. One or more kinds of hydrophilic groups can be immobilized onto the polymer brush. Reactive monomers that have such hydrophilic groups and that are useful in the present invention include, for example, ethanolamine, hydroxyethyl methacrylate, hydroxypropyl acrylate, vinylpyrrolidone, dimethylacrylamide, ethylene glycol monomethacrylate, ethylene glycol monoacrylate, ethylene glycol dimethacrylate, ethylene glycol diacrylate, triethylene glycol diacrylate and triethylene glycol methacrylate. Thus a polymer brush may itself comprise a functional group, or one may be immobilized to the brush.

One or more kinds of functional groups can be immobilized on the polymer brushes. Such groups can be combined or immobilized in discrete multi-layers to impart an additional degree of functionality to the composition. For example, the present invention provides materials having enzymatic activity such as the ability to phosphorylate or dephosphorylate a target polypeptide substrate, the ability to digest, i.e., a nucleic acid at a restriction site, or hydrolyze a polypeptide, the ability to radiolabel a polynucleotide or polypeptide, i.e., using I¹²⁵, I¹³¹, P³², S³⁵, Cr⁵¹ and other radionuclides, or the ability to catalyze a biological or chemical reaction. Examples of enzyme functional groups that can be bound to or isolated using the polymer brushes, and potential uses for those enzymes, include, but are not limited to ascorbic acid oxidase (e.g., for avoidance of interference of ascorbic acid on diagnostic assays of blood, urine, or other samples), aspartase (e.g., for conversion of fumaric acid to L-aspartic acid), aminoacylase (e.g., for conversion of acetyl-D,L-amino acids to L-amino acids), tyrosinase (e.g., for synthesis of tyrosine from phenol, pyruvate and ammonia), lipase (e.g., for hydrolysis of a cyano-ester to ibuprofen or hydrolysis of a diltiazem precursor), penicillin amidase (e.g., for production of ampicillin and amoxycillin), hydantoinase and carbamylase (e.g., for hydrolysis of 5-p-HP-hydantoine to d-p-HP-glycine), DNase (e.g., for hydrolysis of DNA to oligonucleotides), bovine liver catalase (e.g., for hydrolysis of hydrogen peroxide), trypsin and chymotrypsin (e.g., for hydrolysis of whey proteins), arginase and asparaginase (e.g., for hydrolysis of arginine and asparagine), proteases (e.g., to remove organic stains from fabrics), lipases (e.g., to remove greasy stains from fabrics), amylase (e.g., to remove residues of starchy foods from fabrics), cellulase (e.g., to restore a smooth surface to fibers of fabrics and restore fabrics to their original colors), proteases and lipases (e.g., to intensify flavor and accelerate the aging process of foods), lactase (e.g., to produce low-lactose milk and related products for special dietary requirements), beta-glucanase (e.g., to help the clarification process of wines), cellulase (e.g., to aid the breakdown of cell walls in winemaking), pectinase (e.g., to improve fruit-juice extraction and reduce juice viscosity), cellulase (e.g., to improve juice yield and color of fruit juice), lipase (e.g., for hydrolysis of fats and oils or the production of fatty acids, glycerine, fatty acids (e.g., used to produce pharmaceuticals, flavors, fragrances and cosmetics), alpha-amylase (e.g., for liquefaction of starch or fragmentation of gelatinized starch), aminoglucosidase (e.g., for saccharification or complete degradation of starch and dextrins into glucose), alpha-amylase (e.g., for conversion of starch to fructose), glucoamylase and pullulanase (e.g., for saccharification), glucose isomerase (e.g., for isomerization of glucose), beta-glucanase (e.g., for reduction of beta-glucans), beta-glucanase (e.g., for reduction of beta-glucans and pentosans), lipase, amidase and nitrilase (e.g., for manufacture of enantiomeric intermediates for drugs and agrochemicals), lipase (e.g., to remove fats in the de-greasing process in the leather industry), amylase and cellulase (e.g., to produce fibers from less valuable raw materials in the textiles industry), xylanase (e.g., as a bleaching catalyst during pretreatment for the manufacture of bleached pulp for paper), beta-galactosidase (e.g., for hydrolysis of lactose to glucose), trypsin and chymotrypsin (e.g., for hydrolysis of high-molecular-weight protein in milk), alpha-galactosidase and invertase (e.g., for hydrolysis of raffinose), alpha-amylase, beta-amylase, and pullulanase (e.g., for hydrolysis of starch to maltose), pectinase (e.g., for hydrolysis of pectins), endopeptidase (e.g., for hydrolysis of k-casein), protease and papain (e.g., for hydrolysis of collagen and muscle proteins), glucose oxidase and catalase (e.g., for conversion of glucose to gluconic acid), lipase (e.g., for hydrolysis of triglycerides to fatty acids and glycerol, hydrolysis of olive oil triglycerides, hydrolysis of soybean oil, butter oil glycerides and milk fat), cellulase and beta-glucosidase (e.g., for hydrolysis of cellulose to cellobiose and glucose), and fumarase (e.g., for hydrolysis of fumaric acid to 1-malic acid). Alternatively, microorganisms or fragments thereof can be functional groups, for example, such as Pseudomonas dacunhae (e.g., for conversion of L-aspartic acid to L-alanine), Curvularia lunata/Candida simplex (e.g., for conversion of cortexolone to hydrocortisone and prednisolone), or Saccharomyces and other yeasts (e.g., for fermentation of sugars and anaerobic fermentation); all can be immobilized on the polymer brushes.

The functional groups can include all hydrophilic groups, anionically dissociating groups and/or cationically dissociating groups, and enzymes. Stated more specifically, the polymer brush can include multiple functional groups (e.g., anionically dissociating groups and hydrophilic groups, or alternatively cationically dissociating groups and hydrophilic groups) or three kinds of functional groups (e.g., hydrophilic groups, anionically dissociating groups, and cationically dissociating groups), or more (e.g., hydrophilic groups, anionically dissociating groups, cationically dissociating groups, enzymes, SpA and one or more immunoglobulins). Combinations of functional groups that are appropriate in the present invention include, for example, an ionic group and a non-ionic group, i.e., an amine group with a coexisting hydrophilic group. A preferred embodiment additionally comprises a second functional group in combination with the first functional groups described above. In a currently preferred embodiment, the first, second, third, and fourth functional groups are immobilized on the polymer brushes in multilayers. Thus, one of the major features of the present invention is that different kinds of molecules having hydrophilic domains (non-ions) present in a sample solution with molecules having ionic domains (anions and/or cations), or molecules having a phosphorylation state, or a binding site or nucleotide or polypeptide sequence can be recovered, purified, concentrated and isolated, modified, synthesized, or otherwise utilized with the compositions of the invention. The functional group may be altered to change the binding of a substrate bioactive molecule, to thereby tailor the dissociation rate in vivo, and provide controlled release of the substrate bioactive molecule bound thereto. Such alteration or chemical modification may be effectuated on the compositions of the present invention, or the modifications may be effectuated before immobilization to the polymer brush surface.

The functional groups can include antibodies or domains or fragments thereof. Hydroxysuccinimide esters, for example, provide one method for immobilizing one or more antibodies to the polymer brushes via lysine residues, thereby producing functionalized materials with affinities for antigenic targets. The carbohydrate moieties, described above, provide yet another source for immobilization to the polymer brushes or to functional groups. The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site. Thus, an intact antibody has two binding sites. Except in bifunctional or bispecific antibodies, the two binding sites are the same. The chains all exhibit the same general structure of relatively conserved framework regions (FR) joined by three hyper variable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol 196:901-917 (1987); Chothia et al. Nature 342:878-883 (1989). All such domains or fragments or sequences therefrom may be immobilized on polymer brushes by the methods described herein. In one aspect, immobilization of an immunoglobulin to the polymer brushes through the CH2 or CH3 domains is preferred. Without being restricted to theory, it is believed such immobilization decreases steric hindrance and increases the surface area available for antigen binding.

A bispecific or bifunctional antibody is an artificial hybrid antibody having two different heavy/light chain pairs and two different binding sites. These provide for increased functionality of the materials, as two targets can be bound by one immunoglobulin molecule, useful for example, where it is desirable to crosslink two targets, such as in an enzymatic reaction. Bispecific antibodies can be produced by a variety of methods including fusion of hybridomas or linking of Fab′ fragments. See, e.g., Songsivilai & Lachmann Clin. Exp. Immunol. 79:315-321(1990), Kostelnyet al. J. Immunol. 148:1547-1553(1992). Production of bispecific antibodies can be a relatively labor intensive process compared with production of conventional antibodies and yields and degree of purity are generally lower for bispecific antibodies. Bispecific antibodies do not exist in the form of fragments having a single binding site (e.g., Fab, Fab′, and Fv) but a bispecific antibody can be immobilized as described, and provides an additional functional property for the polymer brushes, i.e., an additional specificity for a ligand. Multiple isotypes, species, and epitope recognition properties can be imported to the polymer brushes by the methods described herein.

Humanized, or chimeric antibodies are also appropriate immunoglobulins for developing functionalized materials according to the present invention. Such approaches for generating these are further discussed and delineated in U.S. patent application Ser. No. 07/466,008, filed Jan. 12, 1990, Ser. No. 07/610,515, filed Nov. 8, 1990, Ser. No. 07/919,297, filed Jul. 24, 1992, Ser. No. 07/922,649, filed Jul. 30, 1992, filed Ser. No. 08/031,801, filed Mar. 15, 1993, Ser. No. 08/112,848, filed Aug. 27, 1993, Ser. No. 08/234,145, filed Apr. 28, 1994, Ser. No. 08/376,279, filed Jan. 20, 1995, Ser. No. 08/430, 938, Apr. 27, 1995, Ser. No. 08/464,584, filed Jun. 5, 1995, Ser. No. 08/464,582, filed Jun. 5, 1995, Ser. No. 08/463,191, filed Jun. 5, 1995, Ser. No. 08/462,837, filed Jun. 5, 1995, Ser. No. 08/486,853, filed Jun. 5, 1995, Ser. No. 08/486,857, filed Jun. 5, 1995, Ser. No. 08/486,859, filed Jun. 5, 1995, Ser. No. 08/462,513, filed Jun. 5, 1995, Ser. No. 08/724,752, filed Oct. 2, 1996, and Ser. No. 08/759,620, filed Dec. 3, 1996 and U.S. Pat. Nos. 6,162,963, 6,150,584, 6,114,598, 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998). See also European Patent No., EP 0463 151 B1, grant published Jun. 12, 1996, International Patent Application No., WO 94/02602, published Feb. 3, 1994, International Patent Application No., WO 96/34096, published Oct. 31, 1996, WO 98/24893, published Jun. 11, 1998, WO 00/76310, published Dec. 21, 2000. The disclosures of each of the above-cited patents, applications., and references are hereby incorporated by reference in their entirety. Humanized, or chimeric antibodies or domains or fragments thereof can be immobilized to the polymer brushes as described.

Liposomes, microsponges and microspheres act as functional microdelivery groups, and may be immobilized to the materials described herein. These are useful for delivery of a compound contained within the microdelivery device to a target cell, where the cell is contacted with the functionalized materials having the liposomes, microsponges and microspheres containing the compound, immobilized thereto. Liposomes are lipid molecules formed into a typically spherically shaped arrangement defining aqueous and membranal inner compartments. Liposomes can be used to encapsulate compounds within the inner compartments, and deliver such compounds to desired sites within a cell. The compounds contained by the liposome may be released by the liposome and incorporated into a cell, as for example, by virtue of the similarity of the liposome to the lipid bilayer that makes up the cell membrane. A variety of suitable liposomes may be used, including those available from NeXstar Pharmaceuticals or Liposome, Inc. Liposomes may be immobilized to the polymer brushes by several methods, for example through interactions with the hydrophobic polymer brushes, or by a functional group, for example, a fatty acid functional group. Uses include transfection of cell targets introduced to the functionalized materials having immobilized liposomes with nucleic acids contained within the liposomes.

Microsponges are high surface area polymeric spheres having a network of cavities which may contain compounds. The microsponges are typically synthesized by aqueous suspension polymerization using vinyl and acrylic monomers. The monomers may be mono or difunctional, so that the polymerized spheres may be cross-linked, thus providing shape stability. Process conditions and monomer selection can be varied to tailor properties such as pore volume and solvent swellability, and the microsponges may be synthesized in a controlled range of mean diameters, including small diameters of about 2 micrometers or less. A standard bead composition would be a copolymer of styrene and di-vinyl benzene (DVB). The compounds contained by the polymeric microsponges may be gradually released therefrom due to mechanical or thermal stress or sonication. A variety of suitable microsponges may be used, if functionalized as by the procedures described herein, including those commercially available from Advanced Polymer Systems. Since these are themselves polymers, they can be grafted to the polymer brushes or otherwise immobilized by standard chemical techniques known in the art in view of the teachings described herein. Uses of such functionalized materials having microsponges include introduction of bioactive molecules or compounds contained within the microsponges into cell targets introduced to the functionalized materials.

Bipolar Functionalized Materials

The techniques disclosed herein are applicable for the development of materials that have bipolar properties. These bipolar functionalized materials include materials having, for example, charged functional groups on one region or surface and oppositely charged functional groups on another region or surface. Example One describes the preparation and use of a bipolar membrane, but other embodiments are possible, and the morphology of the devices according to the invention is not limited to preparation of a functionalized membrane. Other materials may be utilized, and selective grafting and immobilization of oppositely charged functional groups and including other functional groups of the present invention can be developed by stepwise grafting and masking techniques.

The bipolar membrane of Example One is substantially planar, having cation-exchange functional groups immobilized on one surface and anion-exchange functional groups immobilized on the opposing surface. The characteristic of this bipolar membrane is that when a voltage is applied, both functional groups at the center phase of the membrane will dissociate water into H⁺ and OH⁻. Without being restricted to theory, there are two assumptions that may explain the mechanism of water dissociation. First, the voltage generated in the center phase of the membrane electrically dissociates water into H⁺ and OH⁻ according to the second Wien effect. Second, the reaction between immobilized charged functional groups with water molecule leads to the dissociation of water into H⁺ and OH⁻ according to a chemical reaction (see, R. Simons, J. Membr. Sci., 78, 13 (1993) and H. Strathmann et al., J. Membr. Sci., 125, 123 (1997) each incorporated herein by reference).

Applications for using such a bipolar functionalized material are numerous. For example, a substrate including graft polymerized brushes with affinity purification functional groups and charged functional groups can be used for high-throughput isolation and subsequent isoelectric separation of polypeptides. In another aspect, a substrate is used for the production of compounds for the pharmaceutical and biotechnology industries, such as salicylic acid. Other applications include but are not limited to, electrodialysis, recycling of salts from waste treatment, production of acid and alkali from concentrated seawater.

Methods of producing bipolar functionalized materials include pasting, casting and plasma graft polymerization methods (see, Iwamoto et al., Nihon Kagaku Kashi, No. 6, 425 (1997) in Japanese, G. S. Trivedi et al., React. Funct. Polym., 28, 243 (1996), Y. Yokoyama et al., J. Membr. Sci., 43, 165 (1989) each incorporated herein by reference). Each have their advantages as would be known to one of skill in the art. The present device illustrated at Example One demonstrates electrolytic performance comparable to casted electroseparation devices, but is believed to be easier and more cost effective to manufacture and operate.

Immobilization of Cells and Microbes to the Functionalized Materials

The materials of the present invention are capable of interaction with a wide range of biological and chemical targets, including nucleic acids, peptides, organic and inorganic molecules and compounds, enzymes and multi-subunit polypeptides and the like. In one aspect, the functionalized materials interact with these targets a and catalyze a reaction. In another aspect, the materials bind the targets. In addition to these small compounds, the functionalized materials are useful for with larger target structures such as polypeptide complexes like multi-subunit enzymes, organelles, membrane receptors and membranes. In another aspect, the materials and devices of the present invention are capable of interacting with targets including intact cells, for example but not limited to, human and animal cells, bacteria, viruses, and fungi. These targets can, in turn, be immobilized to the polymer brushes for use as subsequent functional groups, thereby providing additional levels of functionality to the materials.

Immobilization of larger structures including multi-subunit polypeptides, organelles, cells and microorganisms can be effectuated by many of the numerous types of functional groups disclosed herein. Charged functional groups, for example, tertiary amino functional groups, can be used as highly effective filters to capture viruses and virus particles from liquids with minimal removal of proteins (see, U.S. Patent Application No.: 20010034055 to Lee et al., incorporated herein by reference). Example Two describes the use of quaternary amino functional groups for the immobilization of Staphylococcus aureus bacteria from liquid culture preparations. This bacteria can, in turn, be used to immobilize immunoglobulin molecules through its surface Protein A, or used for other purposes. Applications for isolation of bacteria from solutions are numerous, and include purification techniques. Immobilization of a bacterium on a device of the present invention also includes applications in the field of cloning and expression of recombinant genes and polypeptides. For example, device comprising a membrane capable of immobilizing bacteria and nucleic acids further comprises bipolar functional groups, and can be used for single step electroporation and transformation of the bacteria with the nucleic acids. Another application is the immobilization and transfection of mammalian cells using, for example transfections to create cell lines, hybridomas, and transgenic organisms, where the device comprises polymer brushes having functional groups for the immobilization of cells and nucleic acids, and chemical transfection methods are used to cause uptake of the polynucleotides into the cells.

Many charged molecules on the cellular surfaces are targets for immobilization via charged functional groups. Acidic sugars including those incorporated into many polysaccharides, glycoproteins and glycolipids are potential targets for anion exchange functional groups, such as but not limited to, N-acetylneuraminic acid (sialic acid) or N-acetylmuramic acid. Sialic acid, for example, is found on many human cells including erythrocytes, and can be targeted for immobilizing the same on the materials of the present invention. Cation exchange functional groups are useful in the isolation of cells or microorganisms having surface polysaccharide amino sugars, such as but not limited to, N-acetylglucosamine, N-acetylgalactosamine, galactosamine, and mannosamine. Other compounds such as peptidoglycans, glycosaminoglycans (e.g., chondroitin, keratin, and hyaluronic acid among others), and proteoglycans all display negative charges and can serve as targets for anion exchange functional groups, permitting the binding of cells to the materials of the present invention. Further targets for immobilization of bacteria include lipopolysaccharide (LPS), for example O antigens and H antigens a major component of the membrane of gram negative pathogenic bacteria like E. coli and Salmonella typhimurium. Other pathogenic bacteria, such as Staphylococcus, Streptomyces, Clostridium, Yersinia, and Bacillus can be similarly isolated by the materials of the present invention and evaluated for pathogenicity as described.

Immobilization of cells and microbes need not be based solely on the binding of charged targets to charged functional groups on the polymer brushes. For example, polysaccharides have numerous hydroxyl groups which may serve as targets based on their ability to form hydrogen bonds. In addition, lipids and fatty acids are the major components of biological membranes, and can be immobilized by non-polar functional groups, as well as functional groups comprising lipid anchors, e.g., glycophosphoinosital, phosphatidylserine, sphingomyelin, phosphatidylcholine, and phosphatidylethanolamine, and phosphatidylinosital, among others. In addition, specific surface receptors, integral membrane proteins, peripheral proteins, annexins, and ion channels can provide targets for affinity purification functional groups that can immobilize cells. In a preferred aspect, these affinity purification functional groups include immunoglobulins or fragments thereof, capable of specific binding to targets on the cells. For example, hemagglutinin (HA) and neuraminidase on orthomyxovirus or paramyxovirus virions are targets for anti-HA and anti-neuraminidase antibodies, VPI is a target for immobilization of picornanviruses, and gp120 is a target for the immobilization of HIV virions using an anti-gp120 antibody. Enveloped viruses in particular, are amenable to the targeted separation based on the techniques disclosed herein. In particular, polypeptides in the lipoprotein membrane surrounding poxvirus virions, e.g, variola and vaccinia, provide targets for immobilization of virions by affinity based methods, and are also markers for identification and phenotypic determination. A comprehensive listing of surface antigens for bacteria, for viruses, and for fungi can be found respectively in e.g., Bergy's Manual of Systematic Bacteriology, Field's Virology, and Atlas of Clinical Fungi G. S. De Hoog, et al, each incorporated by reference in their entirety. Other surface antigens on mammalian and in particular human cells, are well know in the medical and biotechnology arts (see, Altered Glycosylation in Tumor Cells (UCLA Symposia on Molecular an Cellular Biology, Vol 79) Christopher L. Reading (Editor), Immune Complexes and Human Cancer (Contemporary Topics in Immunobiology, Vol 15) Fernando A. Salinas, Michael G., Jr. Hanna (Editor), Tumor Markers: Biology and Clinical Applications (Cancer Research Monographs, Vol 4) Nasser Javadpour (Editor), and the American Type Culture Collection catalogue found online at http://www.atcc.org/, each incorporated herein by reference. These surface antigens are useful for targets for immobilization according to the present invention, i.e., based on the affinities of antibodies to the surface antigen, or based on weak covalent interactions, or other physical properties. The selection of particular functional groups for immobilization of cells is thus believed to be routine for one of ordinary skill in the art in view of the targets expressed on the particular cell or organism desired, in view of the teachings provided herein. For example, Bacillus anthraces spore biomarkers can be used as targets to isolate the bacillus from a sample mixture. Samples are obtained from water supplies or from clothing, or tissues of organisms. The sample is introduced to the materials, which have functional groups with affinity for the spore biomarkers, and the isolated organisms are evaluated for pathogenicity, for example, by determination of expression levels of the anthrax toxin components PA (protective antigen), LF (lethal factor) and EF (edema factor), from isolated organisms, see, Elhanany E., et al., Rapid Commun Mass Spectrom 2001; 15(22):2110-6, incorporated by reference, which details isolation of the organism and detection by matrix-assisted laser desorption/ionization time-of-flight mass spectrometry (MALDI-TOFMS). In this aspect, B. anthraces spores are immobilized to the materials, which further include sinapinic acid or alpha-cyano-4-hydroxycinnamic acid immobilized thereto as the matrix, followed by linear mode analysis of the materials.

Polypeptide or oligosaccharide markers are cellular targets and functional groups having affinity thereto are used to immobilize cells to the materials. In one aspect, the materials are used to isolate stem cells based on the presence of surface antigenic determinants, indicating cells committed to a particular lineage, or differentiated immature cells e.g., hematapoetic stem cells from cord blood or bone marrow having one or more of the markers CD10+, CD19+, CD34, +PAX5, E2A, EBF, ATM, PDGFRA, SIAH1, PIM2, C/EBPB, WNT16, and TCL1, each expressed at intervals during B-cell maturation, see, Muschen et al., Proc Natl Acad Sci USA 2002 Jul. 23;99(15):10014-9, incorporated by reference. These are useful for the creation of cell lines, for characterizing cellular development, and such other applications requiring isolated cells. In another aspect, the materials have an affinity for a target such as a marker for a disease state. For example, transcription of CBFI responsive genes lead to the overexpression of CD23, a marker of B-cell chronic lymphocytic leukemia. In this embodiment, the isolated cells are used in subsequent diagnosis or to monitor for therapeutic endpoints in the treatment of a disease characterized by aberrant expression levels of the markers. For example, the cells are immobilized and the presence or absence of the markers is determined and correlated to the presence or absence of a disease state. Many such markers for disease states are known in the medical arts. In a preferred aspect, human cells are immobilized to the materials of the present invention by appropriate functional groups having affinity for such marker targets. Using such isolated cells in routine assays, the phenotypes and genotypes of these isolated cells can be determined and correlated with information about disease states.

The materials of the present invention are inert to a wide variety of reagents and conditions. For example, polypropylene and polyethylene are resistant to acids and alkalis, as well as numerous solvents and denaturants, such as urea, chloroform, formaldehyde and dimethylsulfoxide (DMSO). Advantages of the present materials include applications where carbohydrate, lipid or protein fractions are extracted from the immobilized cells. In addition, the materials can withstand temperature extremes of heating and in particular, freezing temperatures. Thus, the materials are particularly advantageous where it is desirable to immobilize cells and subsequently freeze them, without necessitating an elution step. For example, E. coli cells are immobilized and treated with media including 10% DMSO, then stored at −80 degrees C., directly on the materials in the appropriate cryogenic nutrient solution. Without being restricted to theory, it is believed that viability of the immobilized cell is enhanced as the polymer brushes immobilize the cell in multiple dimensions over its surface, thereby without substantially deforming the cellular membranes.

Functionalized Material Libraries

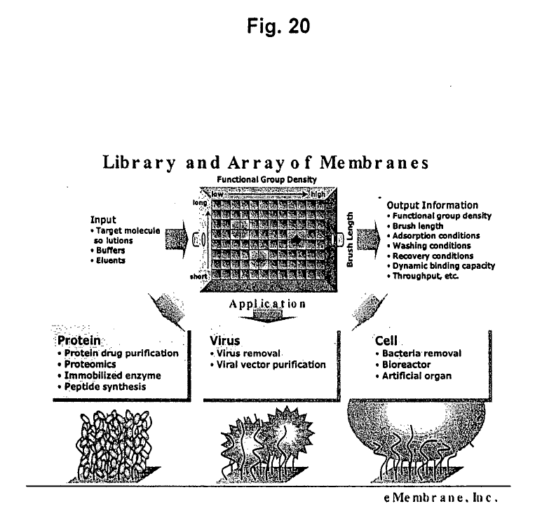

Current graft polymerized materials have been produced using a directed process whereby each material is custom-made and evaluated for a specific biotechnology application. Although successful, this process is laborious and time consuming, and thus limited in its ability to produce new products. To accelerate the discovery process, a library of functionalized materials is needed, having varying properties, for example a plurality of polymer brush domains, each provided as small surfaces (e.g., about 1 micrometer² to about 2 cm 2) that vary over a wide range of physical and functional properties in both the (x) and (y) dimensions. (see, FIG. 20). A “library” or “material library” as used herein, refers to a substrate including one or more surfaces having a plurality of functionalized materials presented thereon. A “domain” as used herein, refers to an independent region of a particular functionalized material. The same functionalized material may be used at more than one domain, thereby providing redundancy in the library. A domain may be in direct proximity to another domain, and may contact it, or the domains may not be in physical contact but may be in fluid communication, or the domains may be isolated. An “address” or “domain address” as used herein, refers to the region or location of a domain within the library, where the location can be described by e.g., Cartesian coordinates or other means of providing a spatial description. Thus, a library comprises a plurality of domains, each having an address.

Similar in concept to the libraries produced by combinatorial chemistry companies for identifying promising drug candidates, the library can be rapidly produced and rapidly screened for optimizing the development of new materials or optimization of existing ones. Among the parameters to be varied include the chemical composition of base polymers, type of monomers used for forming brushes, brush densities, brush lengths, and type and degree of chemical functionalization of polymer brushes. To illustrate the potential complexity of the library, there are well over 1000 different reactive monomers available off-the-shelf that are suitable for forming polymer brush structures using the grafting techniques disclosed herein. Moreover, these monomers can be used in various blends, and can be functionalized using different immobilization chemistries and functional groups to generate an enormous library of unique materials spanning a wide range of functionalities. Furthermore, the process of producing and screening material libraries according to the present invention is suitable for miniaturization and automation.

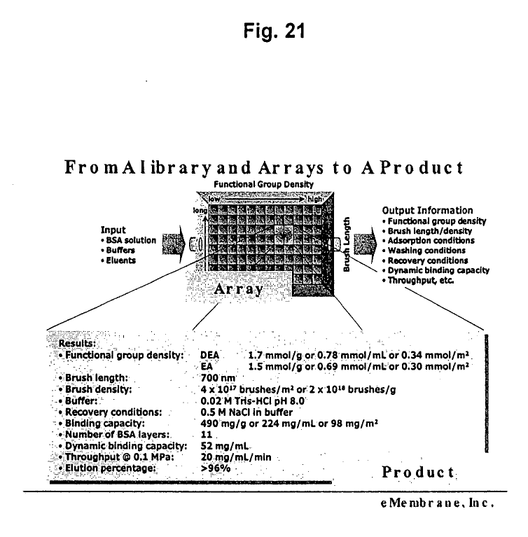

These libraries may further be housed in a microfluidic device that will facilitate the rapid screening of the library using high-throughput methods to identify domains of candidate materials that interact with target molecules (FIG. 21). For example, a membrane for purification of a therapeutic protein is desired. Candidate materials are first selected based on high-throughput screens of a library, for domains of functionalized materials that bind this specific target protein. This screen will identify the optimal composition of material (i.e., material, brush composition, brush length/density and functional group density), based upon binding of the target to one or more domains in the library. In addition, this screen will also determine the appropriate environmental conditions (e.g., salt concentration, temperature, cofactor requirements, etc.) needed for the optimal binding and elution of the target protein. Thus, by performing a high-throughput screen, the library not only identifies a unique material that binds a commercially important protein or biological target, it has also identified the conditions for the optimal use and performance of this material.

To create the library, a plurality of domains of functionalized materials, each varying from the others in terms of its physical or functional properties, is presented or formed on at least one surface of a substrate. One or more of the materials disclosed herein are suitable for use as a substrate, for example, an organic thin film coating on a glass surface provides an appropriate substrate, to which polymer brushes can be grafted and subsequently functionalized. Alternatively, functionalized materials may be prepared and later affixed to the substrate.

The functionalized materials or polymer brushes that are grafted or otherwise affixed to the substrate are presented as independent domains on a surface of the substrate. Through the techniques described herein, brush densities as well as brush length and morphology can be varied. Further, standard masking techniques can be applied, allowing the grafting process to be controlled with respect to selected domains. A material library of the present invention includes at least two domains, and more preferably 10, 50, 100, 1000, or 10,000 domains. In a currently preferred embodiment, the array comprises about 1 domains. Each domain covers a mean surface area of the substrate less than about 0.25 mm². Preferably, the area of the substrate surface covered by each of the domains is between about 1 μm² to about 10,000 μm². In a particularly preferred embodiment, each domain covers an area of the substrate surface from about 100 μm² to about 2,500 μm². The domains of the array may be of any geometric shape. For instance, the domains may be square, rectangular or circular. The domains of the array may also be irregularly shaped. The domains are optionally elevated from the median plane of the underlying substrate. The distance separating the domains of the array can vary. Preferably, the domains of the array are separated from neighboring domains by about 1 μm² to about 5000 μm². Typically, the distance separating the domains is roughly proportional to the diameter or side length of the domains on the array if the domains have dimensions greater than about 10 μm². If the domain size is smaller, then the distance separating the domains will typically be larger than the dimensions of the domain. Each domain has an independent location or domain address, on the substrate. In a currently preferred embodiment, the domains are presented in a two-dimensional matrix format and the address of each domain is described by a set of Cartesian coordinates having the values (x) and (y). Each domain includes one or more functional groups immobilized on the polymer brushes. Functional groups are preferably covalently immobilized on the polymer brushes, either directly or indirectly, at concentrations varying from about 0.001 fg/mm² material surface area to about 100 g/mm² material surface area. The number of different functional groups immobilized at a domain address will vary depending on the application desired. It is possible to create multifunctional domains by using different functional groups, for example, renaturation of a polypeptide and binding to a particular functional group that recognizes the native polypeptide may be accomplished by incorporating into a particular domain, groups capable of hydrolyzing urea in addition to groups that bind the folded polypeptide. Additionally, a series of domain locations with different urease activity levels may be created, and the degree of renaturation required for polypeptide binding may be determined by detecting protein binding at each domain address. Similar arrangements of functionalized materials can evaluate a target's ability to bind or perform a biological function in terms of in other variables, for example but not limited to over a range of pH values or over a range of salt concentrations, or over a range of concentrations of cofactors such as metal ions, phosphates, or antioxidants, or over a concentration range of antagonists or agonists. For example, an antibody to HIV protein gp120 is used as a universal functional group and can capture the virus, immobilizing it to the polymer brushes at each domain. Two candidate binding site antagonists are introduced to the library, varying the concentrations at each domain. Fluorescently labeled CD4 is introduced to the library. Quantitation of CD4 binding to the virus with respect to the combinations of receptor antagonists is evaluated by detection of fluoresence at each domain address. This is related to the degree of antagonist activity, which in turn, is a function of the antagonist concentrations, known for each domain address. Therefore, the optimal antagonist concentrations are determined.

Many combinations of functionalized materials are possible to create a material library. For example, in a single material library each of the domains may be a distinct functionalized material in that each may comprise different functional groups, i.e., a material library having 100 domains could comprise 100 different functionalized materials each with a unique functional group. Likewise, a material library of 10,000 different functionalized materials could comprise 10,000 domains, each having three distinct functional groups not used at any other domain address, leading to a library with an overall complexity of 30,000 different functional groups. In contrast, in a single material library each of the domains may be a distinct functionalized material but may share in part, a common functionality with other domains in that each domain may comprise combinations of similar and different functional groups, i.e., a material library having 100 domains could comprise 100 different functionalized materials, each having a unique first functional group, but half of the domains may share a common second functional group and the remaining half may share a common third functional group. In alternative embodiments, a plurality of domains have common functional groups, i.e., a library has three-thousand domains, but only comprises one thousand different functionalized materials, as each different functional group is immobilized at three common domains. In a currently preferred embodiment, a plurality of domains have common functional groups but each of these domains is still a distinct functionalized material as each varies from the others in terms of polymer brush density, brush length and brush morphology.

The type of functionalized materials and number of domains used to create a material library will depend on the library's prospective use. For instance, if the library is to be used as a diagnostic tool in evaluating the status of a tumor or other diseased tissue in a patient, a library may comprise about 10 different functionalized materials, 5 domains having functional groups with specificity for the target marker proteins whose expression is known to be indicative of the disease condition and 5 domains having functional groups with specificity for target marker proteins whose expression is known to exclude the disease condition. However, if the library is to be used to measure a significant portion of the soluble protein content of a cell, then the library preferably comprises at least about 10,000 different functionalized materials. Alternatively, a more limited proteomics study, such as a study of the abundances of various human transcription factors, for instance, might only require a library of about 100 different functional materials.

The material libraries are useful for a wide range of applications, depending on the types of functionalized materials. They are used as capture agents for binding of particular targets, such as polypeptides, polynucleotides, membranes, organelles, microorganisms, viruses, prions or cells, particularly human cells. To accomplish this, functionalized materials are selected having groups that bind nucleic acids, polypeptides, complexes of polypeptides, receptors, receptor ligands, oligosaccharides and polysaccharides, lipids, prions, bacterial cells, viruses, fungi, and cells. The libraries are also useful for evaluating the chemical or physical properties of targets, or for performing reactions involving targets. Functional materials are selected having groups that are catalytic agents for reactions involving particular targets, for example, restriction digestion of target nucleic acids, proteolytic cleavage of target polypeptides, and other enzymatic reactions involving target lipids or polysaccharides. In one embodiment, the targets are proteins which are all expression products, or fragments thereof, of a cell or population of cells of a single organism. The expression products may be proteins, including peptides, of any size or function. They may be intracellular proteins or extracellular proteins. The expression products may be from a one-celled or multicellular organisms. The organisms may be plants or animals. In a preferred embodiment of the invention, the binding partners are human expression products, or fragments thereof. In one embodiment, the targets include randomly chosen subsets of all the proteins, including peptides, which are expressed by a cell or population of cells in a given organism or a subset of all the fragments of those proteins. The targets of some or all of the functional materials on the library need not necessarily be known. For instance, the different functional materials of the library may together bind a wide range of cellular proteins from a single cell type, many of which are of unknown identity and/or function. In another embodiment the targets of the functional materials are related proteins. The different proteins bound by the functional materials may optionally be members of the same protein family. The different targets of the functional materials may be either functionally related or just suspected of being functionally related. The different proteins bound by the functional materials may also be proteins which share a similarity in structure or sequence or are simply suspected of sharing a similarity in structure or sequence. Likewise, intact cells may be targets of specific functional materials, for example but not limited to, cells expressing a, desired surface antigen may be isolated e.g., CD4+ cells, or cells expressing cancer markers. This can provide for detection of particular phenotypes of cells isolated from patient tissue samples, i.e., evaluation of an immunocompromised patient's T-cell count, or identification of malignant cells in a sample of tumor tissue.

In one aspect, the material library is used in a microfluidics device. The substrate having the library presented thereon is placed in a reaction chamber. The reaction chamber includes a device that houses the substrate and regulates the environment. Methods for regulating the supply (and removal) of reagents to the reaction chamber, as well as the environment of the reaction chamber (e.g., the temperature, and oxidative environment) are incorporated into the reaction chamber using techniques common in the art. Examples of this technology are outlined in: Kricka, Clinical Chem. 44: 2008-2014 (1998); see also U.S. Pat. No. 5,846,727, both incorporated by reference. For example, the substrate is fixed into a reaction chamber and reagents and buffers as well as solutions of targets are pumped into and out-of the reaction chamber through microfluidic ports on either side of the chamber. In one embodiment, the substrate has etched channels in communication with a plurality of domains, thereby permitting the microfluidic ports to be in fluid communication with the desired functionalized materials on the substrate. Complete exchanges of volume can take place rapidly, i.e., within about 1 second and is mediated by electronically controlled valves and pumps that control the flow of solutions through the microfluidic ports. Control of the same is effectuated by an operator directed system, preferably an automated system. Robotic introduction of fluids onto microtiter plates, gene and proteomics chips or arrays is commonly performed to speed mixing of reagents and to enhance experimental throughput, and the present invention is compatible with such robotic systems. More recently, microscale devices for high throughput mixing and assaying of small fluid volumes have been developed, (for example, U.S. Pat. No. 6,046,056 to Parce et al. incorporated by reference). The microfluidics devices are generally suitable for assays relating to the interaction of biological and chemical species, including enzymes and their substrates, ligands and ligand binders, receptors and ligands, antibodies and antibody ligands, as well as many other assays. Because the devices provide the ability to mix fluidic reagents and assay mixing results in a single continuous process, and because minute amounts of reagents can be assayed, these microscale devices represent a fundamental advance for laboratory science.

According to the present invention, the substrate is positioned in an integrated microfluidic system including a microfluidic device. The device has at least a first reaction channel and at least a first reagent introduction channel, typically etched, machined, printed, or otherwise manufactured in or on a surface of the device that will contain the substrate having the material library. Optionally, the device can have a second reaction channel and/or reagent introduction channel, a third reaction channel and/or reagent introduction channel or the like, up to and including hundreds or even thousands of reaction and/or reagent introduction channels. The reaction channel and reagent introduction channels are in fluid communication, i.e., fluid can flow between the channels under selected conditions. The device has a material transport system for controllably transporting a material through and among the reagent introduction channel and reaction channel. For example, the material transport system can include electrokinetic, electroosmotic, electrophoretic or other fluid manipulation aspects (micro-pumps and microvalves, fluid switches, fluid gates, etc.) which permit controlled movement and mixing of fluids. The device also has a fluidic interface in fluid communication with the reagent introduction channel. Such fluidic interfaces optionally include capillaries, channels, pins, pipettors, electropipettors, or the like, for moving fluids, and optionally further include microscopic, spectroscopic, fluid separatory or other aspects. The fluidic interface samples a plurality of reagents or mixtures of reagents from a plurality of sources of reagents or mixtures of reagents and introduces the reagents or mixtures of reagents into the reagent introduction channel. The fluid is thus directed to domains on the substrate, for example by one or more channels in the substrate in communication with one or more domains. Essentially any number of reagents or reagent mixtures can be thus introduced to the substrate by the fluidic interface, depending on the desired application. Because microfluidic manipulations are performed in a partially or fully sealed environment, contamination and fluidic evaporation in the systems are minimized.

A first reagent from the plurality of sources of reagent or mixtures of reagents is selected. A first reagent or mixture of reagents (for example, comprising a target compound and a buffer solution) is introduced into the first reaction channel, and is then introduced to a first domain of the library, whereupon the first reagent or mixture of reagents react with the functional groups immobilized to the polymer brushes of the material at the first domain. This reaction can take a variety of different forms depending on the nature of the reagents. For example, where the reagents bind to one another, such as where the reagents are an antibody or cell receptor and a ligand, or an amino acid and a binding ligand, the reaction results in a bound component such as a bound ligand. Where the reagents are sequencing reagents, a primer extension product results from the reaction. Where the reagents include enzymes and enzyme targets (substrates), a modified form of the enzyme target typically results. Where two reacting chemical reagents are introduced to the functionalized materials, a third product chemical typically results at the applicable domains. In another aspect, a reaction product from a reaction at one or more domains is analyzed. This analysis can take any of a variety of forms, depending on the application. For example, where the product is a primer extension product, the analysis can take the form of separating reactants by size, detecting the sized reactants and translating the resulting information to give the sequence of a template nucleic acid. Similarly, because microscale fluidic devices of the invention are optionally suitable for heating and cooling a reaction, a PCR reaction utilizing PCR reagents (thermostable polymerase, nucleotides, templates, primers, buffers and the like) can be performed and the amplicons detected. Amplified or transcribed nucleic acids obtained from a first domain may be transferred to a second domain for subsequent processing, for example, restriction digestion to determine the presence or absence of a single nucleotide polymorphism, or radiolabeling, or hybridization. Where the reaction results in the formation of a new product, such as an enzyme-substrate product, a chemical species, or an immunological component such as a bound ligand, the product is typically detected by any of a variety of detection techniques, including autoradiography, chemiluminescence microscopy, spectroscopy, or the like.

Based upon the reaction product, a second reagent or mixture of reagents is selected and introduced to the first domain or one or more second domains, as described above. The second reaction product is similarly assessed. For example, where the product is a DNA sequence, a sequencing primer and/or template for extension of available sequence information is selected. Where the product is a new product such as those above, an appropriate second domain may include components such as an enzyme, ligand, antibody, receptor molecule, chemical, or the like, selected to further test the binding or reactive characteristics of the first or second reaction product. The second reagent or mixture of reagents is introduced to the appropriate domain via the first reaction channel, or optionally the second (or third or fourth . . . or nth) reaction channel in the microfluidic device. The results of the analysis of any reaction product can serve as the basis for the selection and analysis of additional reactants and domains for subsequent introduction to the same or different functionalized materials. For example, a single type of DNA template is optionally sequenced in several serial reactions. Alternatively, completing a first sequencing reaction, as outlined above, serves as the basis for selecting additional templates (e.g., overlapping clones, PCR amplicons, or the like, see, U.S. Pat. No. 6,403,338 incorporated herein by reference).

Analysis of Target Interactions with the Library

Detection of target interactions with functional groups at particular domains within the material library can be accomplished by numerous technologies known and described in the art, including detection of binding or detection of enzymatic or functional activity. Hybridization of nucleic acid targets to immobilized nucleic acid or polypeptide functional groups, or binding of a polypeptide target to a functional group at one or more domains can be detected using labeled targets, labeled functional groups, reagents introduced to the same having detectable labels, or combinations of these approaches. A detectable label may include but is not limited to a luminescent compound such as fluorescein, a chromophore, a fluorescent compound, a radioactive isotope or group containing same, or a nonisotopic label, such as an enzyme or dye, a catalyst, a polynucleotide coding for a catalyst or a promoter, horseradish peroxidase (HRP), alkaline phosphatase, a chemiluminescer such as luminol, a coenzyme, an enzyme substrate, a radioactive group, a small organic molecule, an amplifiable polynucleotide sequence, a particle such as latex or carbon particle, metal, crystallite, liposome, cell, etc., which may or may not be further labeled with a dye, catalyst or other detectable group, a terbium chelator such as N-(hydroxyethyl) ethylenediaminetriacetic acid that is capable of detection by delayed fluorescence, and the like. The detectable label may be directly linked to a polypeptide or polynucleotide target or indirectly linked, e.g., by its presence on a partner molecule that interacts with or binds to a target, for example an HRP conjugated antibody. Such labeling is well known in the art. In general, any label that is detectable can be used. Detectable labels thus include, for example but not limited to (i) labels that can be detected directly by virtue of generating a signal, (ii) specific binding pair members that may be detected indirectly by subsequent binding of a target to a functional group, where either the functional group or target or both contain one or more detectable labels.

In one aspect, the detectable labels are fluorescent, for example but not limited to, fluorescence resonance energy transfer pairs. These refer to a pair of fluorophores comprising a donor fluorophore and acceptor fluorophore, wherein the donor fluorophore is capable of transferring resonance energy to the acceptor fluorophore. In other words the emission spectrum of the donor fluorophore overlaps the absorption spectrum of the acceptor fluorophore. In preferred fluorescence resonance energy transfer pairs, the absorption spectrum of the donor fluorophore does not substantially overlap the absorption spectrum of the acceptor fluorophore. Suitable visible light fluorophores (excitation maximum of the donor above about 350 nm) include fluorescein, Lucifer Yellow, acridine Orange, rhodamine and its derivatives, for example tetramethylrhodamine and Texas Red, and fluorescent chelates or cryptates of Europium. A preferred fluorophore is fluorescein. Suitable energy transfer pairs for detectable labels may be found in, for example, Applications of Fluorescence in Immunoassays (I. A. Hemmila, Wiley Interscience, 1991 and “Molecular Probes: Handbook of Fluorescent Probes and Research Chemicals”—Richard P. Haughland—Molecular Probes Inc.) or may be devised by a person of ordinary skill in accordance with energy transfer principles (for example as outlined by J. R. Lakowicz, Principles of Fluorescence Spectroscopy, Plenum, 1983), each incorporated by reference. When used as detectable labels, fluorophores are generally attached to polypeptides via a side chain of an amino acid in the polypeptide chain, or at the N-terminus or C-terminus. Convenient reagents for labeling amino groups include donor/acceptors derivatized with isothiocyanates, active esters, such as succinimidyl esters, of carboxylic acids or sulphonyl halides. These are generally reacted at moderately alkaline pH in an excess over the compound to be labeled (target or functional groups or the like). Convenient reagents for labeling at thiol groups include donor/acceptors derivatized with maleimido or haloacetyl groups. Two derivatives of GFP are useful for FRET standards, based on their spectral properties. The mutant BFP11, constructed by Lossau et al. (Chem. Physics. 213: 1-16, 1996) using combinatorial mutagenesis, contains the mutations F64M/Y66H. BFP11 has blue-shifted excitation and emission maxima relative to wild-type GFP. The mutant RSGFP4 (Delagrave et al., Bio/Technology, 13:151-154, 1995), generated by combinatorial mutagenesis, contains the mutations F64M/S65G/Q69L. RSGFP4 has spectral properties similar to the S65T mutant reported by Heim et al. (Proc. Natl. Acad. Sci. USA 91:12501-12504, 1994). As the acceptor in a FRET pair, both S65T and RSGFP4 are superior to other red-shifted excitation mutants such as RSGFP8 (F64L+S65T), because the latter mutant has significant excitation in the violet.

After introduction of targets to one or more domains and optional washing to remove unreacted or nonspecifically bound targets, the properties of the target following its interaction with specific domains are determined for the whole library or at specified domain address. The detection means will depend on the detectable label used. For example, with radioactively labeled targets, the activity at a domain address is measured through detection of radioactive emissions at that address, and may be further measured against reference standards at other domain addresses to provide quantitative information about target interactions with the materials library. For fluorescent labeled targets, detection at a domain address includes quantitation of color spectra and intensity, determined, for example, using a scanning confocal microscope in photon counting mode. Appropriate scanning devices for confocal microscopy are described by e.g., Trulson et al., U.S. Pat. No. 5,578,832 and Stern et al., U.S. Pat. No. 5,631,734, each incorporated herein by reference. Further examples of particular labels and their detection can be found in U.S. Pat. No. 5,508,178, the relevant disclosure of which is incorporated herein by reference.

Many systems may be used to detect labels and evaluate interactions between targets and functional groups at one or more domains, for example but not limited to, by detection of electromagnetic radiation, x-ray or particle emission, or by optical examination. Detection systems that may be employed with the present libraries include those described in U.S. Pat. No. 5,508,178, the relevant disclosure of which is incorporated herein by reference. A currently preferred method of detecting labels and quantitating reactions at domains is based on epifluorescence microscopy using FRET as detailed in U.S. Pat. No. 6,456,734 to Youvan et al., hereby incorporated by reference in its entirety, which discloses imaging hardware, software, calibrants, and methods to visualize and quantitate the amount of fluorescence resonance energy transfer (FRET) occurring between donor and acceptor labels. Such a detection system is applicable to the libraries of the present invention when the donor and acceptor labels are affixed to targets and to functional groups or reagents as described above.

Where colormetric or photon based detectable labels are used, an optical reader or other such imaging device is used to read the library. A suitable optical imaging device provides a means for acquiring spatially co-registered electronic images, thus determining detection values for labels at each domain address. Alternatively, the optical imaging device provides a means for reading an impression of the library, e.g., an autoradiograph or chemiluminescent image of the domains of the library. For example, one optical imaging device includes a microscope and digital camera. The microscope can be a steady-state, wavelength-scanning fluorescence microscope (i.e., not a time-resolved system or not an interferometer-based system). The optical imaging device can also provide for background subtraction, spectral overlap corrections, and transformation of data from three channels set into a color space defined by the primary colors of red, green, and blue. Labels first detected at domain addresses and rendered as images with such an imaging device may be further processed to produce an enhanced image, for example, an image in which FRET, acceptor, and donor pixels are more clearly differentiated and pseudocolored. An optical imaging device may include a light source, to provide illumination of the library or for excitation of detectable labels, for example, a 75 watt quartz tungsten halogen (QTH) light source can be coupled directly to the fluorescence microscope, although other light sources may be used if desired. The method of the invention is not limited to an epifluorescence microscope. Macroscopic lens-based systems could be used in place of the microscope's objectives to achieve detection and quantitation over a macroscopic field of view, where the library is large or where the individual domains are easily visualized.

Systems for Evaluating Material Libraries

To control the application of targets and reagents to the material library, and to detect and quantify interactions between the targets and the functionalized materials and relate such interactions to domain addresses, a computer system is utilized. Further, to interpret the information obtained from using the material libraries, and to control and to guide the use and evolution of the libraries, the computer system includes one or more programs in informatics. In addition to cataloging the composition of, and physical and functional properties of each material used at particular domains in the libraries, the informatics program will track the target detection data obtained from all uses involving a particular material. This information is used to direct future assays involving related target molecules to or domains of similar function or composition, thus accelerating the discovery process. Moreover, these data sets will also guide new directions for the expansion of the library, i.e., the evolution of materials with specific properties. For example, a high-throughput screen might determine that the optimal binding/elution of a particular polyclonal antibody target occurs with materials containing a narrow range of brush densities or lengths and that this occurs at domains where there are specific blends of monomers that contain certain concentrations of ionic functional groups and immunospecific (antigenic) functional groups. The library thus can be used to indicate the optimal materials for isolating immunoglobulins having specific affinity ranges for the antigen. Lastly, the informatics program is used to develop sophisticated molecular models of the polymer brushes that will simulate brush structure/function by taking into account critical factors such as brush composition, density, length and flexibility. When combined with the data sets obtained from the target screens, these molecular models will help predict binding properties of the polymer brushes, thus speeding the discovery process and guiding continued expansion of new functionalized materials for the library.

In one aspect, the systems of the present invention provides an integrated computer program that compares digital profiles of images of library domains and causes the system to select one or more addresses, and generates instructions that direct a robotic device to isolate reaction products from the domains. In one embodiment, the system directs the robotic device to introduce these reaction products to secondary domains and directs the microfluidic device to introduce reagents to the secondary domains. In yet a further embodiment, the system includes a library information management system that tracks materials used in the libraries and tracks data associated with targets, such as clinical data, operations performed on the targets, and data generated by analysis of the reactions of the targets and materials.

According to the present invention, targets are introduced to a library comprising functionalized materials presented as domains having addresses, as described above. The interactions of targets with functional groups is detected and evaluated by a reading means, such as the optical imaging device described. The reading means thus provides both qualitative and quantitative information about the interaction of targets to functional groups through detection of labels at domain addresses on the library, which is communicated to computer system. The system includes an instruction set having modules for data management, e.g., a data input means, a data storage means, a data retrieval means, a relational database, and a data output means, as well as an instruction set comprising data analysis algorithms e.g., detection and quantitation of labels. The instruction set may further include control modules, e.g., for control of robotic or microfluidic devices. Processors appropriate for executing the instruction set of the system include any processors capable of recognizing an instruction set written in an appropriate language, for example but not limited to PowerPC based Apple® computers, Pentium® or similar PC type computers, SUN® or Silicon Graphics® workstations, or systems running LINUX or UNIX. The instruction set includes a computer readable algorithm for data analysis, which is stored in computer readable media as part of a program written in a suitable computer readable language, for example C, C++, UNIX, FORTRAN, BASIC, PASCAL, or the like. The program provides the processor with instructions for performing calculations on the input data, as well as other functional elements contained in one or more modules or subroutines (e.g., relational database capabilities, search features, and other user defined functions). The program includes input modules for entering data into the system in computer readable format and a selection module instructing the system to select and read data entered. In one aspect, the system includes an input module. Users of the system enter data into the system in computer readable format, which can be stored in RAM or ROM, or a more permanent storage medium such as a disk or tape drive. The information entered through the input module is thus accessible to the system processor. The system further comprises an output module. The output of the computer system can be represented on a display or monitor, as a word processing text file, formatted in commercially-available software such as WordPerfect® and Microsoft Word®, or represented in the form of an ASCII file, stored in a database application, such as DB2, Sybase, Oracle, or the like. A skilled artisan can readily adapt any number of data processor structuring formats (e.g. text file or database) in order to obtain computer readable medium having recorded thereon the expression information of the present invention.

Computer assisted data analysis minimally includes detection of the labels and determination of signal intensity for each domain address, rendered as an image of the domain address, and where the address is further given as a set of two-dimensional Cartesian coordinates. Where multiple detectable labels are used, i.e., for FRET, a set of three such images from each channel (donor, acceptor, and FRET) can then be processed as three spatially coregistered images or treated as a single image in which each pixel has three color space coordinates corresponding to the monochrome wavelengths. In one embodiment, the quantitative method of the invention performs quantitative FRET measurements and analyses. FRET image data is obtained with standard filter sets using a fluorescence microscope and processed as described above. Equations for quantitative FRET useful in designing specific data analysis algorithms for the program are provided in Gordon, et al., Quantitative Fluorescence Resonance Energy Transfer Measurements Using Fluorescence Microscopy, Biophysical Journal, Vol. 74, May 1998 2702:2713, which is hereby incorporated by reference. In a preferred embodiment, the data analysis further includes information about the materials and the reactions occurring and detected at each domain location, i.e., number and type of functional groups, polymer brush density and morphology, and reaction conditions such as flow rate, target concentrations, salt concentrations, buffer composition, ionic strength, temperature, pH, and time, binding and affinity or catalytic activity. In a more preferred embodiment, the data analysis includes information obtained from or stored within a database, that contains information about the targets, such as their source and method of isolation, and correlation with a disease state.

The invention thus provides a computer-generated digital profile representing the identity and relative abundance of a plurality of target biomolecules detected in view of a plurality of reaction conditions, thereby permitting computer-mediated comparison of profiles from multiple target samples for multiple materials. This automatable technology for screening biological targets and comparing their profiles permits rapid and efficient identification of individual targets whose presence, absence or altered expression is associated with a disease or condition of interest. Such targets are useful in the design and evaluation of their potential as therapeutic agents, as targets for therapeutic intervention, and as markers for diagnosis, prognosis, and evaluating response to treatment. This technology also permits rapid and efficient identification of sets of targets whose pattern of expression is associated with a disease or condition of interest; such sets of targets provide constellations of markers for diagnosis, prognosis, and evaluating response to treatment.

As those skilled in the art will recognize, the invention described herein can be modified to accommodate and/or comply with any one or more computer based technologies and standards. In addition, variations, modifications, and other implementations of what is described herein can occur to those of ordinary skill in the art without departing from the spirit and the scope of the invention as claimed. Further, virtually any aspect of the embodiments of the invention described herein can be implemented using software, hardware, or in a combination of hardware and software.

### EXAMPLE ONE

### Method and Process for Producing Bipolar Membranes

This following example describes a new method for preparing bipolar functionalized materials, in this case, fabricated in the form of a membrane. Using this method, polymer brushes that contain cation-exchange and anion-exchange functional groups are introduced separately on opposing surfaces of a membrane in a single step. Electron-beam treated high density polyethylene film (HDPE) was used as the material for grafting, and was sandwiched between a monomer solution containing sulfonic acid groups and a monomer solution containing quaternary ammonium salt groups. X-ray microanalysis (XMA), was used to measure the distribution of sulfur and chlorine across the membrane thickness, i.e., the distribution of cation-exchange and anion-exchange groups, respectively. The bipolar membrane was used for electrodialysis to regenerate HCl and NaOH from a NaCl solution. This bipolar membrane demonstrates similar electricolytic potential as compared to bipolar membranes prepared by conventional methods.

Preparation of a Bipolar Membrane and its Characters

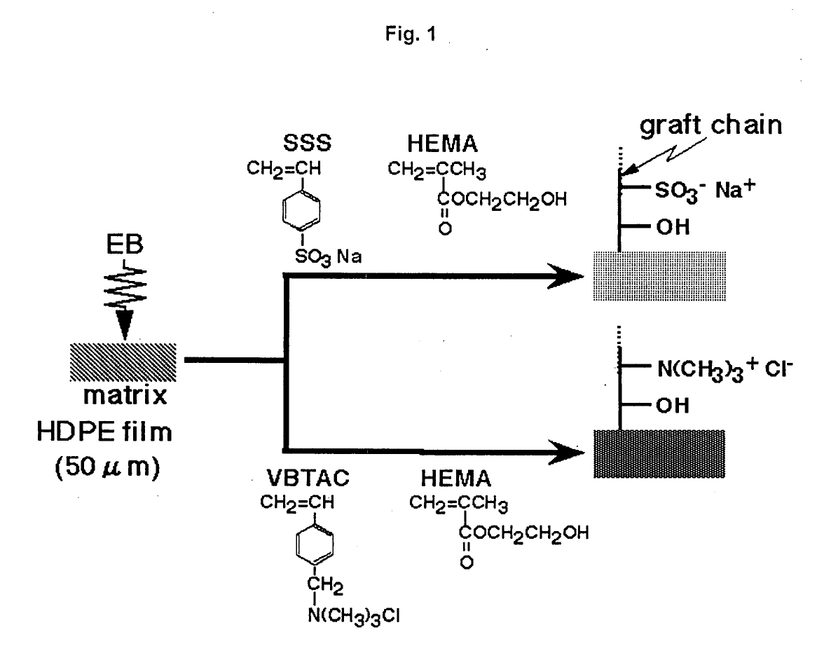

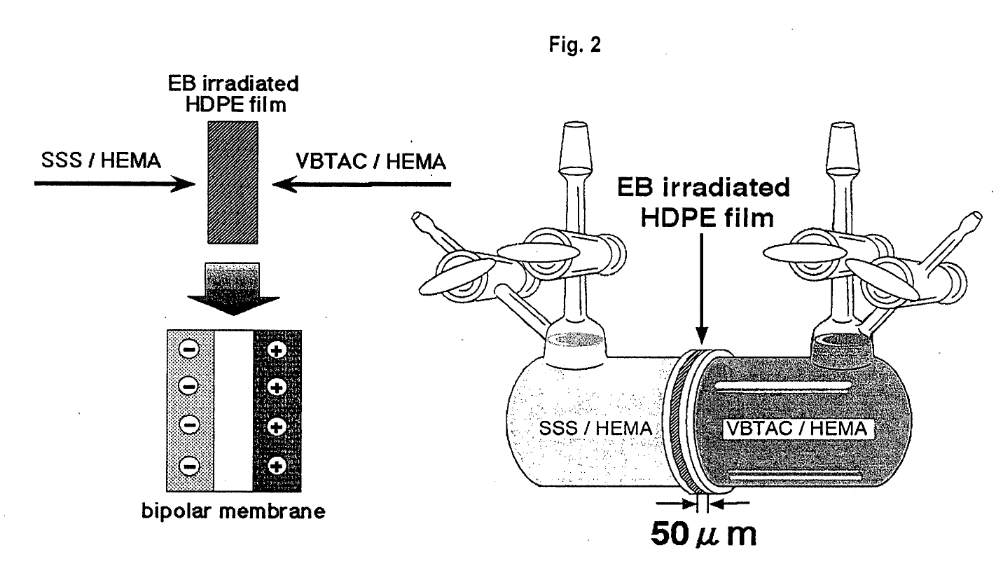

In order to determine the conditions to produce a bipolar membrane, ion-exchange membranes were prepared as shown in FIG. 1. A nonporous high density polyethylene film (HDPE) with a thickness of 50 micrometers was used as the polymeric base membrane for grafting. The base membrane was irradiated with electron beams at a total dose of 200 kGy. As shown in FIG. 2, the electron-beam treated HDPE film was interposed between two identical cylindrical cells having an inner diameter about 4 cm.

Sodium styrene sulfonate (CH₂═CHC₆H₄SO₃Na; SSS) and 2-hydroxylethyl methacrylate (CH₂═CCH₃COOCH₂CH₂OH; HEMA) were co-grafted onto the electron-beam-treated base membrane to form a cation-exchange membrane (see, S. Tsuneda et al., J. Electrochem. Soc., 142, 3659 (1995) incorporated herein by reference). Similarly, vinyl benzyl trimethyl ammonium chloride (CH₂═CHC₆H₄CH₂N(CH₃)₃Cl; VBTAC) and HEMA were co-grafted to form an anion-exchange membrane (see, W. Lee et al., J. Membr. Sci., 81, 295 (1993) incorporated herein by reference). All reaction conditions are listed in Table 1, below. The degree of co-grafting (dg) is defined as:
Degree of co-grafting (dg)=(weight of grafted polymer brushes)/(weight of base membrane)×100%

TABLE 1
Preparation Conditions for Bipolar Ion-Exchange Membranes

| | Cation-exchange | Anion-exchange |
|---|---|---|
| Base polymer | HDPE film | |
| Total irradiation dose | 200 kGy | |
| Monomer | SSS/HEMA | VBTAC/HEMA |
| Solvent | Water | DMF/MeOH = 1/1 vol. |
| | | ratio |

| | |
|---|---|
| Reaction time | 1˜9 h |
| Reaction temperature | 323 K |

The sulfonic acid (—SO₃H) group density and the quaternary ammonium salt (—N(CH₃)₃⁺Cl⁻) group density of the prepared membrane were determined by methods known in the art for salt-splitting capacity, and the hydroxyl group density was calculated by subtracting the ion-exchange group density from the total functional group density. The sulfur (S) profile of the sulfonic acid group and the chlorine (Cl) profile of the quaternary ammonium salt group across the thickness of the prepared membranes were determined by an electron-probe X-ray microanalysis (XMA). In order to measure the electrical resistance of the bipolar membrane, the membrane was sandwiched between two identical cylindrical cells having an effective membrane area of approximately 7 cm² with the quaternary ammonium salt group side facing the anode and the sulfonic acid group side facing the cathode. The electrical current was measured across a 0.1 M NaCl solution at 298 K, using an applied voltage of 15 V.

Electrodialysis Using a Bipolar Membrane

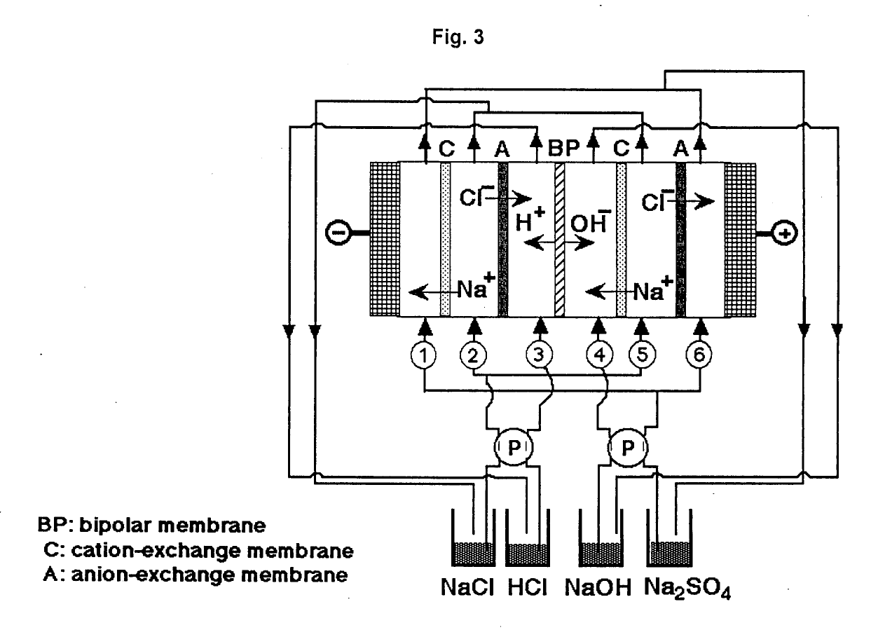

As shown in FIG. 3, with the bipolar membrane placed in the center, conventional cation-exchange and anion-exchange membranes (2 membranes each) were placed in between the electrodes in a electrodialysis chamber. The quaternary ammonium salt group side of the bipolar membranes faced the anode. A voltage of 5.1 V was applied. The operation conditions for the electrodialysis are summarized in Table 2, below. The effective membrane area of all membranes was 10 cm². The NaCl concentration in the salt chambers (2 and 5) was determined by measuring the conductivity. The HCl concentration in the acid chamber (3) and the NaOH concentration in the alkali chamber (4) were measured by titration of acid and base. The concentrations of sodium, chlorine and sulfonic acid ions before and after the electrodialysis were determined by ion chromatography. The electric power unit was determined using the equation:
electric power unit (Wh/kg)=∫V i dt/w

where V, I, t and w are the voltage (V), current (A), time (h) and weight of removed NaCl (kg), respectively.

TABLE 2
Measurement Conditions of Electrodialysis

| | |
|---|---|
| Applied voltage | 5.1 V |
| Duration | 5 h |
| Salt chamber | 1.0 M NaCl |
| Acid chamber | 1.0 M HCl |
| Alkali chamber | 1.0 M NaOH |
| Electrode chamber | 0.5 M Na₂SO₄ |
| Circulation rate | 50 mL/h |
| Temperature | 298 K |

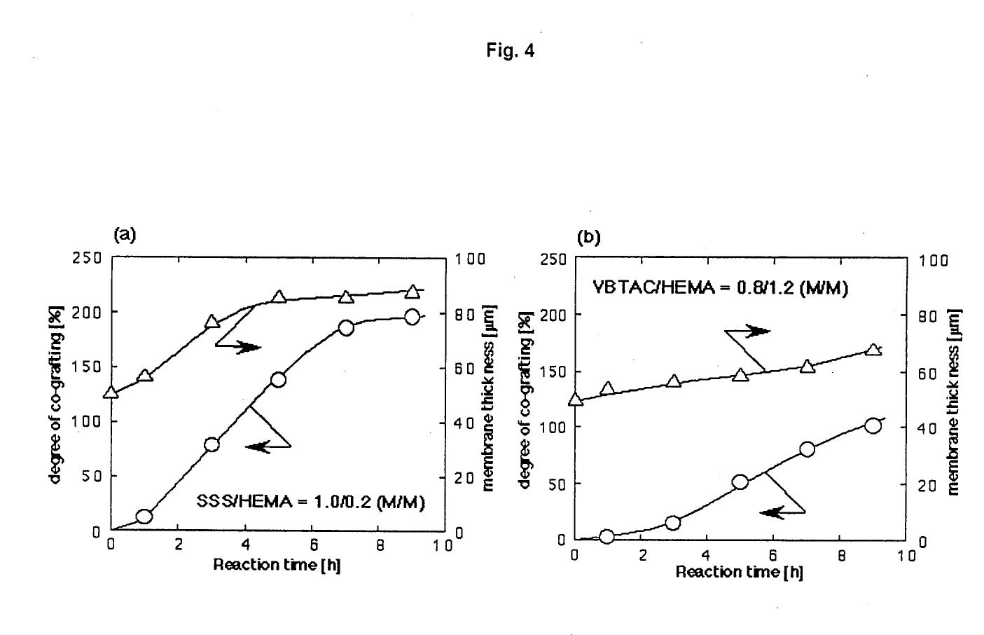

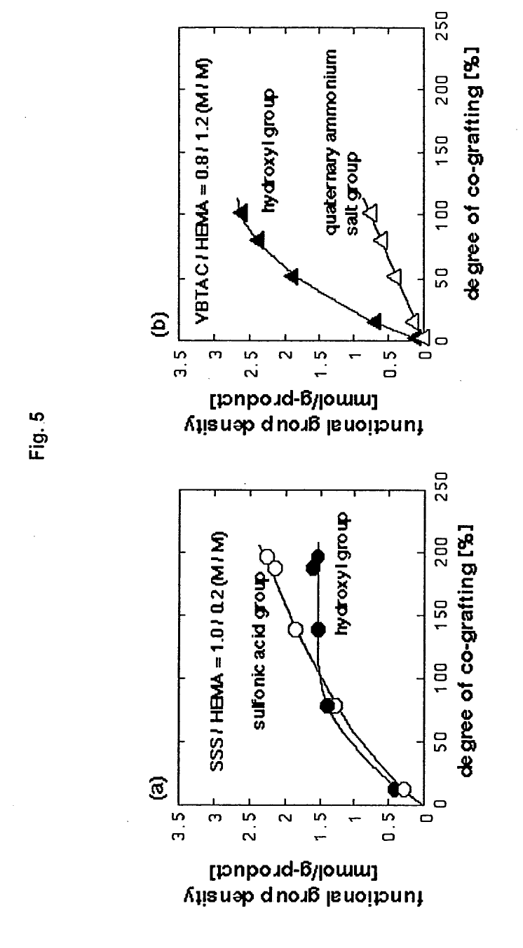

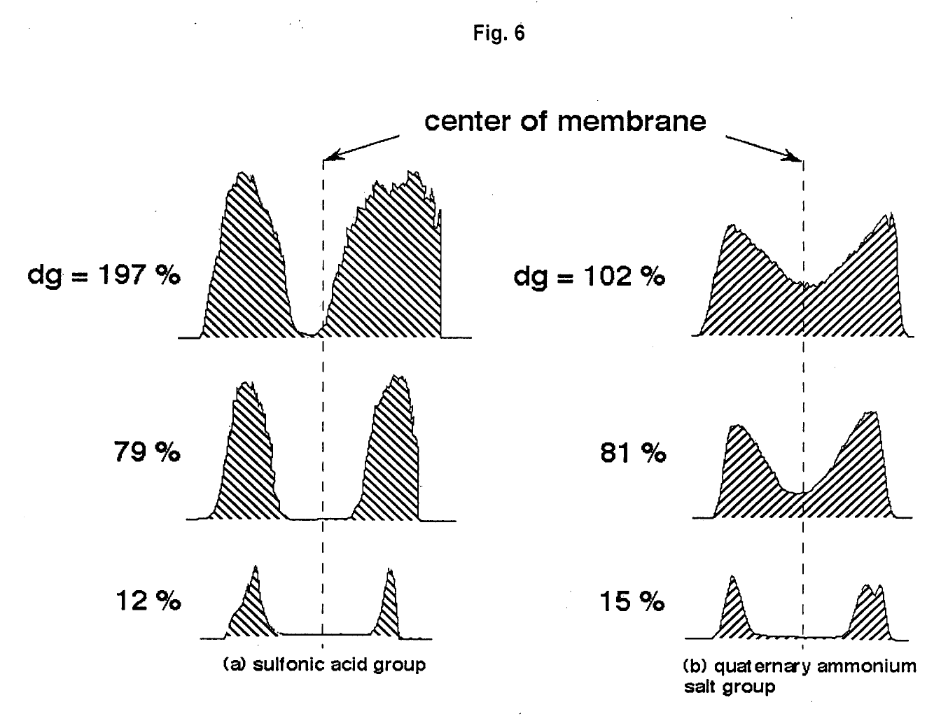

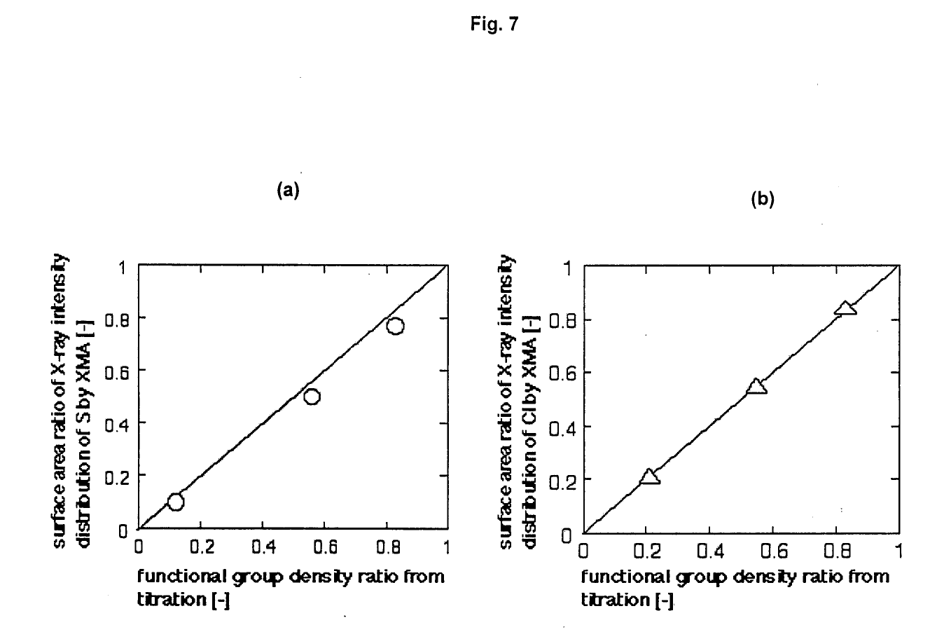

The degrees of co-grafting of SSS/HEMA and VBTAC/HEMA onto the electron-beam-treated HDPE base membranes and their changes in membrane thickness as a function of co-grafting time are shown in FIG. 4. The degree of co-grafting increased with the reaction time. The thickness of the prepared cation-exchange membrane (Na-type) and the anion-exchange membrane (Cl-type) increased to a maximum of 1.8-fold and 1.4-fold, respectively. FIG. 5 shows the increase of sulfonic acid group and hydroxyl group densities in the cation-exchange membrane as well as the increase of quaternary ammonium salt group and hydroxyl group densities as a function of degree of co-grafting (dg). At a reaction time of 9 hours, the sulfonic acid group and the quaternary ammonium group densities reached 2.3 and 0.81 mmol/g-product, respectively. By XMA, the profiles of sulfonic acid group (measuring the sulfur) and the quaternary ammonium salt group (measuring the chlorine) across the thickness of both membranes were measured (FIG. 6). The surface area ratio of X-ray intensity distribution was compared to the functional group density ratio from salt-splitting capacity measurement (FIG. 7). Both ratios matched each other to a maximum ratio. This also means that XMA can be used for quantification. With the increase of the reaction time, i.e., the increase of degree of co-grafting (dg), the brushes that contain sulfonic acid group or quaternary ammonium salt group invaded the membrane from both sides and reached the center.

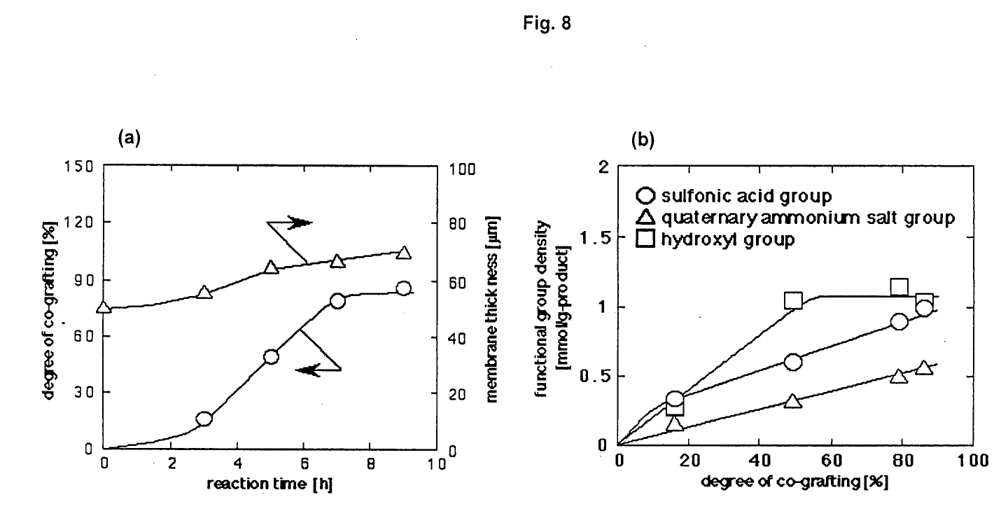

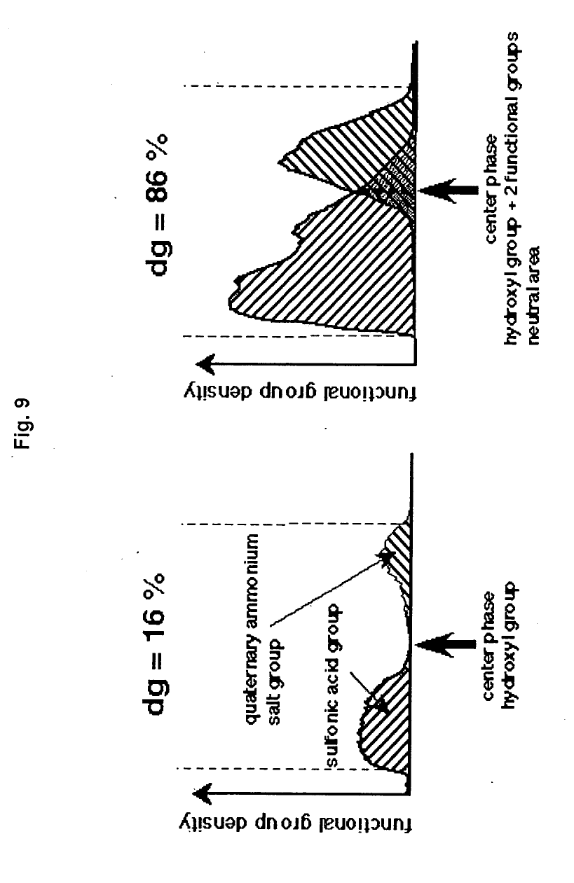

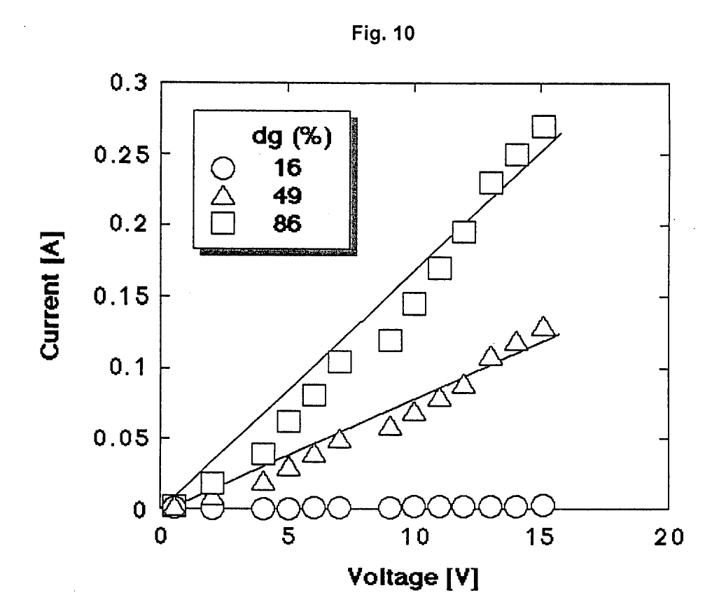

FIG. 8a shows the increase of the degree of co-grafting and the membrane thickness of the prepared bipolar membrane as a function of reaction time. The functional group density also increased with the increase of dg (FIG. 8b). The sulfonic acid group density was about 2-fold of the quaternary ammonium salt group density within the range of dg from 16% to 86%. For example, at dg=86%, the sulfonic acid group density and the quaternary ammonium salt group density were 1.0 and 0.57 mmol/g-product, respectively. The functional group density profiles across the thickness of the dg=16% and dg=86% membranes were compared (FIG. 9). Both membranes have shown the invasion of sulfonic acid groups from the left and the invasion of quaternary ammonium salt groups from the right of the membrane. At dg=16%, both functional groups did not reach the center of the membrane. However, from an observation by scanning electron microscopy (SEM), results have shown that the poly-HEMA brushes have invaded the center of the membrane. In contrast, at dg=86%, both functional groups intersected each other forming a neutral area in the center of the bipolar membrane. The voltage-current characteristic of the prepared bipolar membrane is shown in FIG. 10. At dg=16%, no flow of current was observed even with the increase of voltage. This corresponds to the XMA result that showed no intersection of the functional groups.

Regeneration of Acid and Alkalifrom a Salt Solution

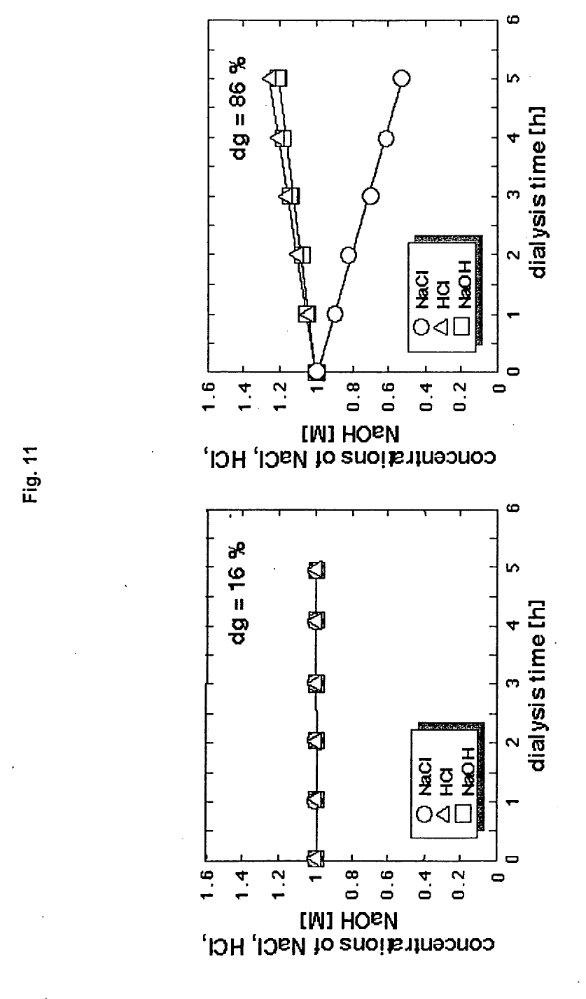

The bipolar membranes with dg's of 16% and 86% were used for electrodialysis. FIG. 11 shows the time course for the concentration changes of NaCl (salt chamber), HCl (acid chamber) and NaOH (alkali chamber). In 5 hours of electrodialysis, M molar of HCl and M molar of NaOH were regenerated from 1 M of NaCl solution. Table 3, below summarizes the mass balance of all ions before and after the electrodialysis of the prepared bipolar membrane (dg=86%). The molar numbers of Na⁺ and SO₄²⁻ agreed with each other before and after the electrodialysis. However, the molar number of Cl⁻ before the electrodialysis did not agree with the one after. Without being restricted to theory, this is believed to be due to the electrode reaction. Calculations using Faraday's law indicate that 3.40×10⁻³ mol of Cl⁻ was needed for the electrode reaction. This value agrees with the shortage of 3.25×10⁻³ mol of Cl⁻ seen before and after electrodialysis.

TABLE 3
Mass Balance of Ions Before and After Electrodialysis of the Co-
Grafted-type Bipolar Membrane (dg = 86%)

| | Before electrodialysis (0 min) | | | | | After electrodialysis (300 min) | | | | |
|---|---|---|---|---|---|---|---|---|---|---|
| | H⁺ | Na⁺ | OH⁻ | Cl⁻ | SO₄²⁻ | H⁺ | Na⁺ | OH⁻ | Cl⁻ | SO₄²⁻ |
| | (10⁻² mol) | | | | | (10⁻² mol) | | | | |
| Salt | | 1.000 | | 1.000 | | | 0.424 | | 0.424 | 0.024 |
| chamber | | | | | | | | | | |
| Acid | 1.000 | | | 1.000 | | 1.262 | 0.016 | | 1.231 | |
| chamber | | | | | | | | | | |
| Alkali | | 1.000 | 1.000 | | | | 1.220 | 1.235 | 0.010 | |
| chamber | | | | | | | | | | |
| Electrode | | 3.000 | | | 1.500 | | 3.320 | | 0.010 | 1.470 |
| chamber | | | | | | | | | | |
| Total | 1.000 | 5.000 | 1.000 | 2.000 | 1.500 | 1.262 | 4.980 | 1.235 | 1.675 | 1.494 |

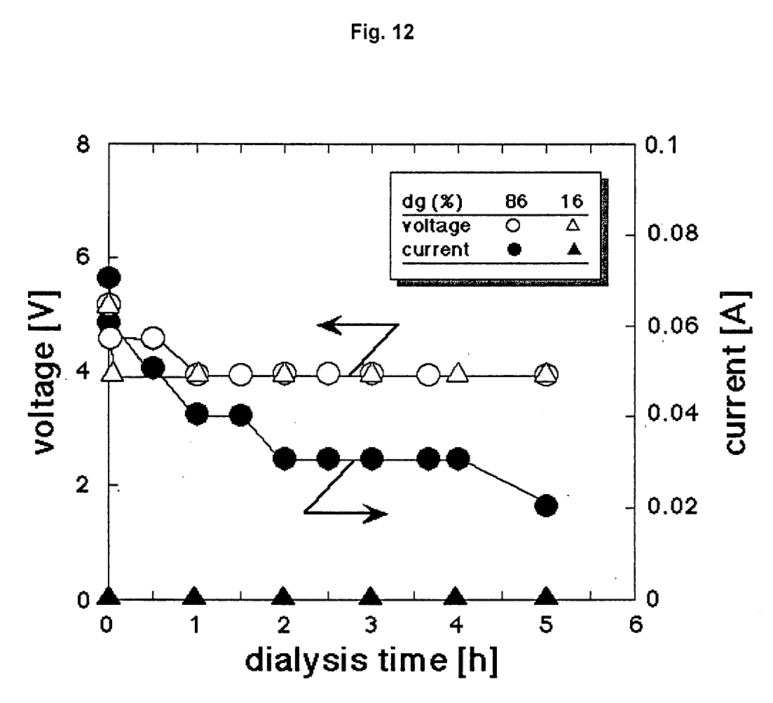

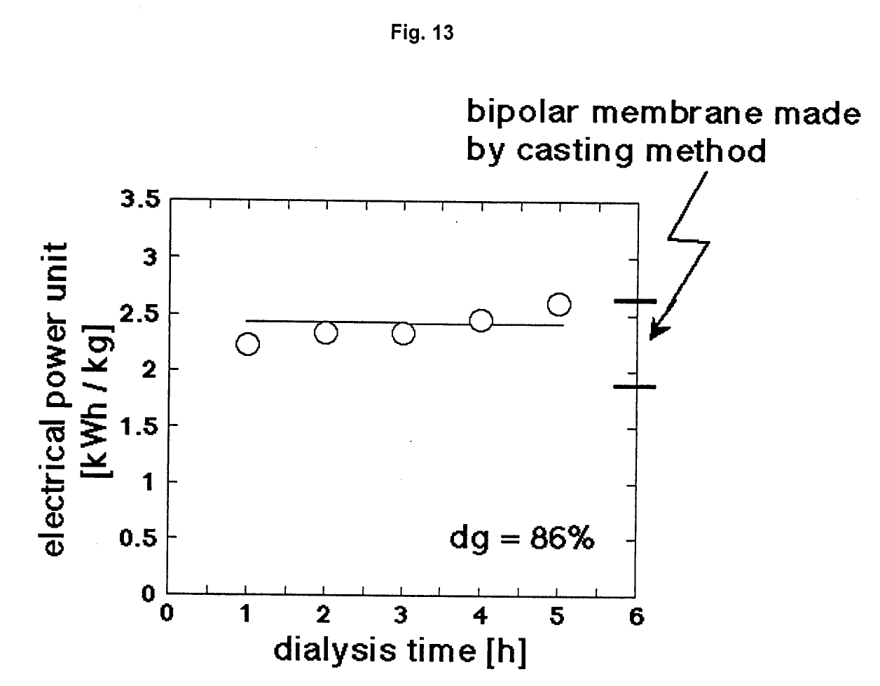

The voltage and current of electrodialysis as a function of operation time showed that the current decreased from the initial 0.07 A to 0.02 A (FIG. 12). The electrical power unit calculated in FIGS. 11 and 12 using the above equation is shown in FIG. 13. The electrical power unit was within the range of 2.2˜2.6 kWh/kg. The electrical power unit obtained by radiation induced graft polymerization (RGIP) was compared to those of previous studies involving pasting or grafting, summarized in Table 4, below, (see, J. Kassotis et al., J. Electrochem. Sci., 131, 2810 (1984), S. K. Adhikary et al., React. Polym., 1, 197 (1983), F. Alvarez et al., J. Membr. Sci., 123, 61 (1997) and G. S. Trivedi et al., React. Funct. Polym., 32, 209 (1997), each incorporated herein by reference). The electrical power unit of the prepared bipolar membrane exhibited similar order of unit (1.6˜2.6 kWh/kg) to the membrane prepared by a casting method.

TABLE 4
Previous Studies of Bipolar Membranes

| | Functional group | | | | | | Electric |
|---|---|---|---|---|---|---|---|
| | density (mmol/g- | | | | | Alkali | power unit |
| | product) | | | | Acid regen. | regen. | (kWh/kg- |
| Prep. | SO₃Na | N(CH₃)₃Cl | | | rate × 10⁻² | rate × 10⁻² | salt |
| method | group | group | (V) | Salt | (M/min) | (M/min) | removed) |
| Pasting | — | — | 3.8 | CH₃COONa | 0.025 | 0.097 | — |
| Pasting | — | — | 50 | Na₂SO₄ | 0.037 | 0.071 | 7.1 |
| Pasting | — | — | 25 | C₆H₄(OH)COONa | 4.00 g/l-min | — | — |
| Casting | 1.33 | 1.00 | 25 | Na₂SO₄ | 0.440 | 0.650 | 2.6 |
| Casting | 1.56 | 1.41 | 20 | Na₂SO₄ | 0.570 | 1.300 | 1.6 |
| Casting | 1.32 | 1.25 | 25 | CH₃COONa | 0.930 | 1.000 | 1.7 |
| RIGP | 1.00 | 0.57 | 5.1 | NaCl | 0.087 | 0.070 | 2.2˜2.6 |

### EXAMPLE TWO

### Immobilization of Microbes Using Graft Polymerized Materials

Preparation of the GMA-DEA-BC Fiber

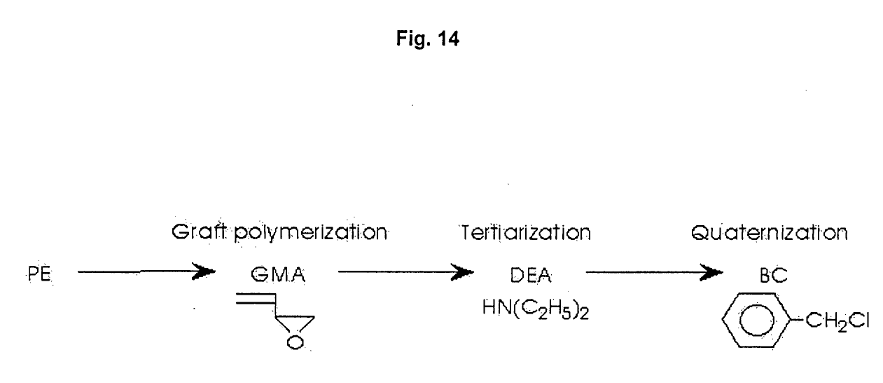

This example describes the immobilization of microbes, in particular the bacteria Staphylococcus aureus, to a material comprising a polyethylene hollow-fiber membrane. The inner and outer diameters of the membrane were 1.9 and 3.2 mm, respectively, with a porosity of 70% and a pore size of 0.34 micrometers. FIG. 14 shows the preparation scheme. A vinyl monomer, glycidyl methacrylate (GMA, CH₂═CCH₃COOCH₂CHOCH₂) was grafted onto the PE membrane by the electron-beam-induced grafting method. The PE membrane was irradiated with an electron beam at a total dose of 200 kGy at room temperature. The irradiated membrane was then immersed in a GMA solution (10% vol/vol in methanol) and reacted at 313 K. The amount of GMA grafted onto the backbone membrane was calculated as the degree of GMA grafting, dg, using the equation provided below.

The GMA-grafted membrane was reacted with diethylamine (DEA, HN(C₂H₅)₂), and then quaternized with benzyl chloride (BC, C₆H₅CH₂Cl). The resulting strong base anion-exchange membranes are referred to as GMA-DEA-BC (dg/Xt/Xq), where dg, Xt and Xq in parentheses designate degree of grafting, conversions of introduction of the tertiary amino group and subsequent quaternization, respectively. The values of dg, Xt and Xq were calculated from the weight change as follows:
dg=100 ((W₁−W₀)/W₀) [%]
Xₜ=100(((W₂−W₁)/73)/((W₁−W₀)/142))[%]
X_{q}=100(((W₃−W₂)/127)/(W₂−W₁)/73))[%]
where W₀, W₁, W₂ and W₃ are the weights of the starting film, GMA-grafted, DEA-introduced and quaternized membranes, respectively. The FIGS. 142, 73 and 127 correspond to the molecular weights of GMA, DEA and BC, respectively.

Culture of Microorganisms

Staphylococcus aureus strain IFO 12732 was obtained from the Institute for Fermentation, Osaka, Japan, and was used as a model microorganism for microbial-cell-immobilization studies. One loopful of the bacteria was inoculated into 10 mL of rehydration fluid (polypeptone 1.0%, yeast extract 0.2%, MgSO₄.7H₂O 0.1%, pH 7.0) and cultured at 305 K for 18 to 24 h in a test-tube shaker at 100 strokes/min. After the cells in the cultured cell suspension were collected by centrifugation at 5600×g for 15 min in a refrigerated centrifuge at a temperature below 277 K to arrest cell division and hold the cells in the stationary phase, and then washed twice with 10 mL of distilled, deionized and sterilized water. The cells were then resuspended in fresh sterilized water to a final volume of 10 mL. The cells were serially diluted in sterilized water to the desired cell concentration (about 10⁵ to about 10⁶ cells/mL) before contact with the prepared grafted membranes.

Microbial Cell Adsorption onto the Grafted-Type GMA-DEA-BC Membranes

Forty milliliters of the prepared cell suspension was added into a 100 mL flask. Then, prepared GMA-DEA and GMA-DEA-BC membranes were brought into contact with the cells by shaking the flask at 130 rpm at 298 K. GMA-DEA and GMA-DEA-BC membranes of 10-cm length (cut into 1-cm sections and subsequently sliced vertically in half) were used. One-tenth milliliter of the contact suspension was pipetted from the flask at specific time intervals, inoculated into 9.9 mL of sterilized water, and serially diluted with sterilized water. One-tenth milliliter of the diluted suspension was spread on an agar plate containing growth media. The plate was incubated at 310 K for 18 to 20 h, and the number of viable cells in the contact suspension was calculated from the number of colonies formed on the plate.

The adsorption rate of microbial cells by both the GMA-DEA and GMA-DEA-BC membranes was examined in terms of the decrease in the number of free viable cells. Values of the adsorption rate constant, taking into consideration of the contact surface area for the microbial-cell-capturing action, were determined from the slopes of the logarithm of viable cell number (CFU/mL; colony forming units per milliliter) versus contact time plots. The adsorption rate constant, k, of the various DEA-EO membranes and DEA beads was defined as:
V(dC/dt)=−kAC
where k can be derived as follows under an initial condition of C=C₀ at t=0, and the adsorption rate constant is defined by:
k=−(V/A)(l/t)ln(C/C₀)[m/s]
where V, A, t, C and C₀ are the volume of viable cell suspension, the contact surface area, the contact time, the viable cell number at contact time t and the initial viable cell number, respectively.

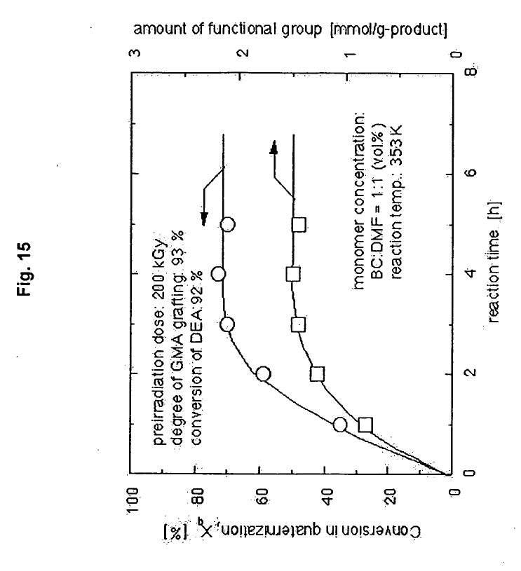

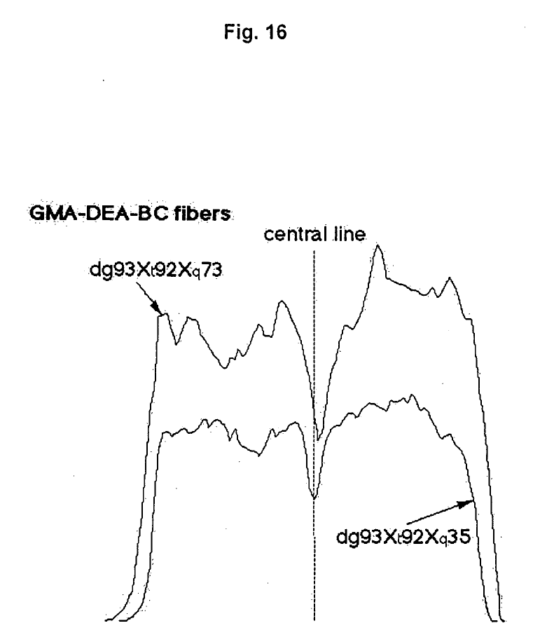

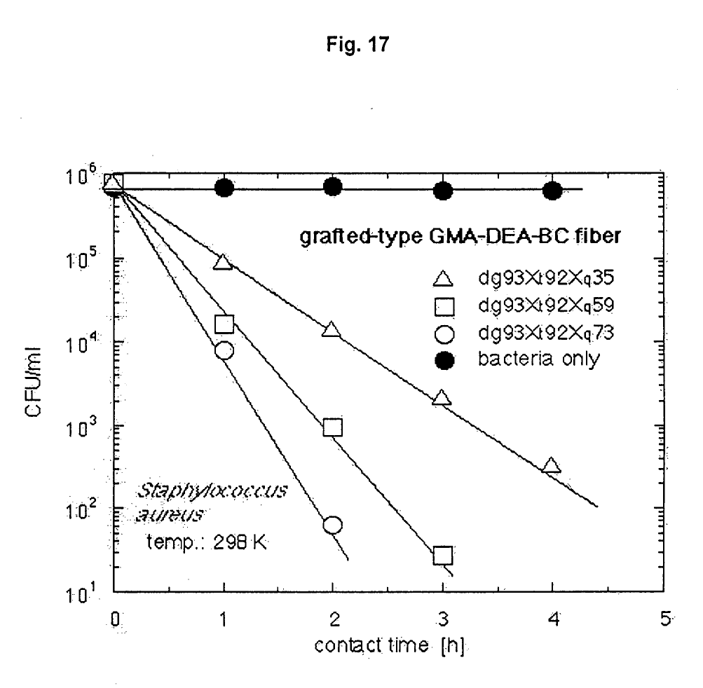

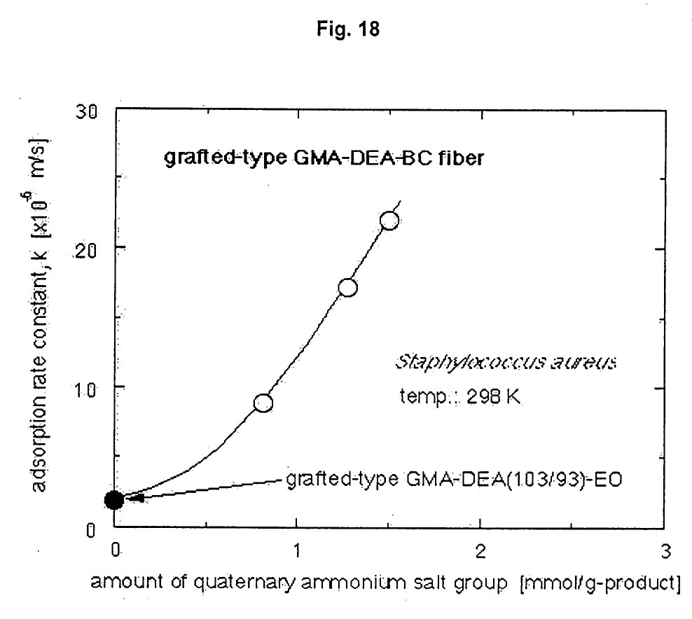

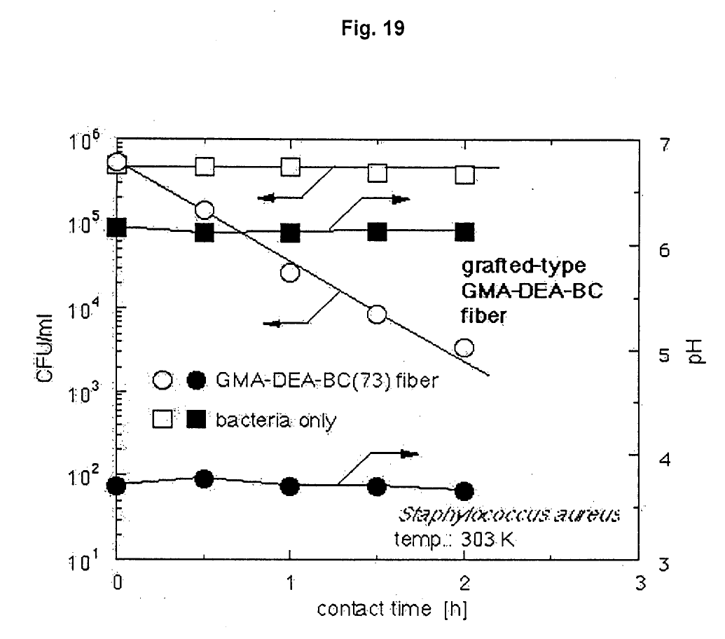

Since the pore size of the membrane is about 0.3 micrometer, S. aureus, which has an average diameter of about 1 micrometer, cannot enter the pores of the membrane. As a result, the contact surface area of the fiber was calculated from the outside surface area only excluding the surface area of the pore. In this experiment excess surface area of membrane was provided (maximum cell coverage of 1%) in order to calculate k. FIG. 15 shows the conversion of quaternization of the grafted-type GMA-DEA-BC fiber. FIG. 16 shows the X-ray microanalysis (XMA) profiles of chloride ion adsorbed on the grafted-type GMA-DEA-BC fibers as a function of conversion of BC. The fibers were converted into Cl-form before the performance of XMA. FIG. 17 shows the adsorption experiments for the grafted-type GMA-DEA-BC fibers against Staphylococcus aureus cells. FIG. 18 shows the relationship between the adsorption rate constant and functional-group-density of the grafted-type GMA-DEA-BC fiber. FIG. 19 shows the changes of CFU/mL and pH when the grafted-type GMA-DEA-BC fiber was brought contact with Staphylococcus aureus cells a function of contact time.

The materials and methods disclosed above are also applicable for the creation of bacterial and viral libraries where, for example, variant strains are immobilized at different domains. Such libraries are useful for detection and characterization of microbial samples, e.g., identification of a pathological strain. In addition, the libraries can be used in the production of antibiotics, for example, to determine the effectiveness of an antibiotic contacted to the bacteria at particular domains at varying dilutions.

### EXAMPLE THREE

### Library of Cells Used for Diagnostic Assays

Libraries of cells from one or more different tissue sources or types are created. These are used, for example, to determine the levels of disease markers in a sample tissue, or for toxicological assays such as for detecting targets in the environment. This example describes the manufacture and use of a human ovarian cell library, for diagnosis of ovarian cancer.

Preparation of the Library

A glass slide having a coating of high density polyethylene film (HDPE) of about 0.5 micrometers in thickness was used as the base material for grafting. The substrate was irradiated with electron beams from a cascade-type accelerator in a nitrogen atmosphere at a total dose of 200 kGy. Glycidyl methacrylate (GMA) was grafted onto the HDPE film by immersion of the substrate in a 10 vol/vol % GMA/1-butanol solution with the grafting reaction temperature at 313 K, to form polymer brushes. After 10 minutes, the substrate was removed and washed to remove any residual GMA and poly-GMA homopolymers. Degree of grafting was calculated as described, and determined to be about 200%. Masking of the substrate prior to grafting permitted the formation of 100 domains of polymer brushes, each having dimensions of 2 mm², presented in a 5×20 matrix pattern.

MUC1 is expressed on the surface of ovarian cancer cells. Nine different splice variants of MUC1 have been described. Obermair, et al., (Int J Cancer 2002 Jul. 10; 100(2): 166-71, incorporated by reference) compares patterns of expression of MUC1 splice variants of malignant and benign ovarian tumors. In this study, ovarian tissue samples were taken from patients with benign ovarian tumors (n=34) and from patients who had surgery for primary (n=47) or recurrent (n=8) ovarian cancer. RT-PCR for MUC1 splice variants A, B, C, D, X, Y, Z, REP and SEC was performed and their expression compared to clinical and histopathologic parameters. Variants A, D, X, Y and Z were more frequently expressed in malignant than in benign tumors. All primary ovarian cancer cases were positive for variant REP but negative for variant SEC. Expression of MUC1 splice variants A, D, X, Y, Z and REP is associated with the presence of malignancy, whereas expression of MUC1/SEC is associated with the absence of malignancy.

Goodheart et al, (Gynecol Oncol 2002 July;86(1):85-90, incorporated by reference) showed that an ovarian cancer p53 mutation is associated with tumor microvessel density, as measured by CD31 staining, and other histopathologic factors. In this study, histopathologic and mutational data were related to CD31 staining utilizing the Mantel correlation statistic. The microvessel density was scored by averaging counts from three high-power (200×) fields. The mean microvessel density counts based on CD31 staining (vessels/HPF) for each FIGO stage and mutation type are reported as: Stage 1 (10.2), Stage 11 (10.7), Stage III (13.8), Stage 1V (22.0), wild-type p53 (9.3), missense p53 mutation (14.4), and null p53 mutation (23.1) with a significant correlation between microvessel density count and FIGO stage (P=0.026), grade (P=0.04), and p53 mutation type (P=0.02).

Lindgren et al., (Int J Oncol 2002 September;21(3):583-9, incorporated by reference) showed that the pattern of estradiol and progesterone differs in serum and tissues of benign and malignant ovarian tumors. Production of both estradiol and progesterone by ovarian cancers has been demonstrated and can be detected. In this study, ovarian tissue, ovarian tumor cyst fluid, ovarian vein samples and peripheral serum concentrations of estradiol and progesterone in pre- and post-menopausal women, subdivided into groups with normal ovaries, benign, borderline and malignant ovarian tumors, were quantitatively assessed. Both ovarian tissue concentrations of estradiol and progesterone were more than 100-fold higher than in serum. Lower concentrations of estradiol, but not progesterone, were found in ovarian cancer tissue, ovarian cyst fluid and peripheral serum in patients with FIGO stages 3 and 4 than in stages 1 and 2. Finding a large ovarian tissue to serum difference of both estradiol and progesterone indicates an important role of ovarian tissue concentrations in tumor biology and can influence anti-hormonal therapy in women with ovarian cancer.

Ovarian cells obtained from tissue biopsies are immobilized to each domain at constant cell numbers. Reference tissues are obtained from ATCC, selected as control cells in view of the detectable markers disclosed. The cells from the patient and reference tissues are immobilized to the brushes at each domain by lipid rich functional groups. Immunoglobulins specific to MUC-1 isoforms A, D, X, Y, Z, REP, and SEC were developed using the methods of Takeuchi et al., (Immunol Methods 2002 Dec. 15;270(2):199, incorporated by reference). Monoclonal preparations of anti-CD31, anti-bcl-2, anti-estradiol, and anti-progesterone immunoglobulins were obtained from commercial sources. The immunoglobulins are fluorescently labeled and applied to the cells at different domains and over concentrations ranging from about 0.001 mg protein/ml to about 10 mg protein/ml. Detection of labeled targets at each domain is performed by confocal microscopy.

Anti-estradiol, and anti-progesterone immunoglobulins were each prepared in serial dilutions to concentrations of antibodies ranging from 15 mg/ml to 1.5 ng/ml in 0.025 M borate buffer (pH about 10). Ten microliters of each of the dilutions were placed on independent domains of the library (each having a mean surface area of about 1 mm²) and reacted with the epoxy groups of the GMA grafted brushes for 24 hours at room temperature, after which the unbound antibodies were removed by washing. Ovarian cells obtained from patient and reference tissues were pulse labeled with ³⁵S and cell lysates were contacted to the domains of the library. The domains were washed, and activity at each domain was assayed by counting the beta emissions at each domain address. The data was interpreted using standard ELISA computer algorithms. The data obtained using both libraries are compared to reference standards and predictive values for these markers known in the medical literature, and a diagnosis is made in view of such information.

### EXAMPLE FOUR

### Library of Randomized Peptides

A library is developed as described, having approximately 10,000 domains. The library is manufactured on a substrate adapted for a microfluidic chamber. Glycine is immobilized to the polymer brushes at each domain, and the unbound glycine removed by washing. The substrate is placed in an automated microfluidic device, and random 6-mer polypeptides are synthesized at each domain, with the peptide sequence at each domain address monitored by the controller computer system. The resultant peptide library is used for screening pulse labeled cell lysates as described. Interactions are detected by counting beta emissions at each domain address. Bound targets at domain addresses are recovered and identified by MALDI-TOF spectroscopy. Data pertaining to the interactions and identification of targets is tracked by a laboratory information management system (LIMS).

### Equivalents

From the foregoing detailed description of the specific embodiments of the invention, it should be apparent that a unique compositions comprising graft polymerized materials having functional groups immobilized thereto in multiple layers, as well as methods of making and using such compositions, has been described. Although particular embodiments have been disclosed herein in detail, this has been done by way of example for purposes of illustration only, and is not intended to be limiting with respect to the scope of the appended claims that follow. In particular, it is contemplated by the inventor that substitutions, alterations, and modifications may be made to the invention without departing from the spirit and scope of the invention as defined by the claims. For instance, the number and kind of functional group combinations, or the use of such compositions in particular devices is believed to be matter of routine for a person of ordinary skill in the art with knowledge of the embodiments described herein.

1. A device comprising a substrate material having polymer brushes on at least a first and a second surface, wherein the polymer brushes on the first surface further comprise a first set of functional groups having a charge, and the polymer brushes on the second surface further comprise a second set of functional groups having an opposite charge to the first set of functional groups.

2. The device of claim 1, wherein the polymer brushes are formed by radical induced polymerization of the substrate material and the degree of grafting of the polymer brushes is greater than 15%.

3. The device of claim 1, wherein the polymer brushes are formed by radical induced polymerization of the substrate material and the degree of grafting of the polymer brushes is greater than 85%.

4. The device of claim 3, wherein the material is bipolar and is capable of dissociating water into H⁺ and OH⁻ when a voltage is applied.

5. A device comprising a plurality of functionalized materials, further comprising
a. a substrate material having at least one surface having polymer brushes formed thereon, the polymer brushes presented in a plurality of domains; and
b. one or more functional groups, the functional groups immobilized to the polymer brushes at one or more domains.

6. The device of claim 5, wherein the polymer brushes at a plurality of domains are of different morphologies or lengths.

7. The device of claim 5, wherein the polymer brushes at a plurality of domains are formed from one or more types of reactive monomers.

8. The device of claim 5, wherein the polymer brushes at a plurality of domains have a degree of grafting from about 10% to about 500%.

9. The device of claim 5, wherein at least one functional group is immobilized at each domain.

10. The device of claim 5, wherein at least two functional groups are immobilized at each domain.

11. The device of claim 5, wherein the functional groups immobilized to the polymer brushes bind one or more targets.

12. The device of claim 11, wherein the targets are selected from the group consisting of polynucleotide targets, polypeptide targets, polysaccharide targets, lipid targets, cell targets, human cell targets, cancer cell targets, animal cell targets, mammalian cell targets, viral cell targets, fungal cell targets, organelle targets, cellular membrane targets and bacterial cell targets.

13. The device of claim 11, wherein the functional groups comprise immunoglobulins or antigen binding fragments thereof.

14. The device of claim 5, wherein the functional groups catalyze a reaction involving a target.

15. The device of claim 14, wherein the functional groups are enzymes selected from the group consisting of restriction enzymes, nucleic acid modifying enzymes, proteases, kinases, and phosphatases.

16. The device of claim 5, wherein the functional groups are charged.

17. The device of claim 12, wherein the bacterial cells are pathogenic strains.

18. The device of claim 17, wherein the bacterial cells are Staphylococcus cells.

19. The device of claim 5, wherein the functional groups are microdelivery functional groups further comprising a compound.

20. The device of claim 19, wherein the target cell is contacted to microdelivery functional groups at one or more domains, thereby causing the target cell to uptake one or more compounds contained in the microdelivery functional groups.

21. The device of claim 20, wherein the one or more compounds are siRNA or antisense nucleic acids.

22. A device comprising a plurality of functionalized materials, further comprising
a. a substrate material having at least one surface having polymer brushes formed thereon by radiation induced graft polymerization, the polymer brushes presented in a plurality of domains; and
b. one or more functional groups, the functional groups immobilized to the polymer brushes at one or more domains.

23. A method of making a library of functionalized materials comprising the steps of:
a. obtaining a substrate material;
b. forming polymer brushes on the material in a plurality of domains; and
c. immobilizing at least one functional group to the polymer brushes at one or more domains.

24. The method of claim 23, wherein the polymer brushes at a plurality of domains are of different morphologies or lengths.

25. The method of claim 23, wherein the polymer brushes at a plurality of domains are formed from one or more types of reactive monomers.

26. The method of claim 23, wherein the polymer brushes at a plurality of domains have a degree of grafting from about 10% to about 500%.

27. The method of claim 23, wherein the functional groups are enzymes selected from the group consisting of restriction enzymes, nucleic acid modifying enzymes, proteases, kinases, and phosphatases.

28. The method of claim 23, wherein the functional groups have an affinity for a target.

29. The method of claim 23 or claim 28, wherein the functional groups or the targets are immunoglobulins or antigen binding fragments thereof.

30. The method of claim 28, wherein the targets are selected from the group consisting of polynucleotide targets, polypeptide targets, polysaccharide targets, lipid targets, cell targets, human cell targets, cancer cell targets, animal cell targets, mammalian cell targets, viral cell targets, fungal cell targets, organelle targets, cellular membrane targets and bacterial cell targets.

31. The method of claim 30, wherein the bacterial cell target is a pathogenic bacterial strain.

32. The method of claim 23, wherein the functional groups are charged.

33. A method of making a bipolar device comprising the steps of:
a. obtaining a material;
b. forming polymer brushes on the material by graft induced polymerization on at least a first and second surface;
c. immobilizing a first functional group to the polymer brushes on the first surface, wherein the first functional group has a charge, and
d. immobilizing a second set of functional groups to the polymer brushes on the second surface, wherein the second functional group has a charge opposite to that of the first functional group.

34. The method of claim 33, wherein the polymer brushes have a degree of grafting greater than 15%.

35. The method of claim 33, wherein the polymer brushes have a degree of grafting greater than 85%.

36. A method comprising using the bipolar material of claim 1 to produce a compound.

37. The method of claim 36 wherein the compound is salicylic acid.

38. A method comprising using the bipolar material of claim 1 to produce acid or alkali from a salt solution.

39. A method comprising using the bipolar material of claim 1 for an electrodialysis reaction.

40. A method comprising using the device of claim 5 to immobilize a target.

41. The method of claim 40, wherein one or more functional groups has an affinity for the target.

42. The method of claim 40, wherein the functional groups comprise an immunoglobulin or antigen binding fragment thereof.

43. The method of claim 40, wherein the functional groups comprise ion-exchange functional groups.

44. A method comprising using the device of claim 5 to catalyze a reaction involving a target.

45. The method of claim 44, wherein the functional groups are enzymes selected from the group consisting of restriction enzymes, nucleic acid modifying enzymes, proteases, kinases, and phosphatases.

46. A method comprising using the device of claim 5 to introduce a compound to a target.

47. The method of claim 46, wherein the functional groups comprise microdelivery groups containing one or more compounds.

48. The method of claim 47, wherein the compound is a nucleic acid, and a cell target is contacted at one or more domains, thereby delivering the nucleic acid to the cell.

49. The method of claim 47, wherein the compound is a polypeptide, and a cell target is contacted at one or more domains, thereby delivering the polypeptide to the cell.

50. The method of claim 47, wherein the compound is a nucleic acid, and a cell target is contacted at one or more domains, thereby delivering the nucleic acid to the cell.

51. The method of claim 47, wherein the compound is a drug, and a cell target is contacted at one or more domains, thereby delivering the drug to the cell.

52. A method of detecting a target comprising the steps of contacting one or more domains of a material library with a solution comprising a target, wherein the target is capable of interacting with functional groups at the domains and wherein a detectable label indicates the interaction, and detecting the label at one or more domains thereby detecting the interaction.

53. The method of claim 52, wherein the interaction between the target and a domain is detected by measuring radioactive emissions at the domain.

54. The method of claim 52, wherein the interaction between the target and a domain is detected by measuring luminescence at the domain.

55. The method of claim 54, wherein an antibody, fragment or derivative thereof is used to detect the interaction.

56. The method of claim 52, wherein the interaction between the target and a domain is detected by measuring fluorescence at the domain.

57. The method of claim 56, wherein the fluorescence measured is generated by fluorescence resonance energy transfer pairs.

58. A system comprising:
a. a processor in communication with one or more memory devices;
b. a material library further comprising a substrate material having at least one surface having polymer brushes formed thereon, the polymer brushes presented in a plurality of domains; and one or more functional groups, the functional groups immobilized to the polymer brushes at one or more of the domains;
c. a reading device capable of detecting labels at library domain addresses, the reading device in communication with the processor;
d. an instruction set stored in at least one memory device, the instruction set capable of interacting with the processor;
e. a user controlled input device capable of entering information into the memory device; and
f. an output device in communication with the processor or memory.

59. The system of claim 58, wherein the functional groups comprise polypeptide sequences.

60. The system of claim 58, wherein the functional groups comprise polynucleotide sequences.

61. The system of claim 58, wherein the functional groups comprise an immunoglobulin or antigen binding fragment thereof.

62. The system of claim 61, wherein the immunoglobulin concentration varies at each domain from about 0.1 fg/mm² antibody immobilized per domain surface area to about 100 mg/mm² antibody immobilized per domain surface area.

63. The system of claim 58, wherein the functional groups comprise human cells.

64. The system of claim 58, wherein the functional groups comprise viral cells.

65. The system of claim 58, wherein the functional groups comprise bacterial cells.

66. A system comprising:
a. a processor in communication with one or more memory devices;
b. a material library further comprising a substrate material having at least one surface having polymer brushes formed thereon, the polymer brushes presented in a plurality of domains; and one or more functional groups, the functional groups immobilized to the polymer brushes at one or more of the domains;
c. a reading device capable of detecting labels at library domain addresses, the reading device in communication with the processor;
d. an instruction set stored in at least one memory device, the instruction set capable of interacting with the processor;
e. a user controlled input device capable of entering information into the memory device;
f. an output device in communication with the processor or memory; and
g. a microfluidic device, wherein the library is contained within a reaction chamber of the microfluidic device.

67. The system of claim 66, wherein the processor is in communication with one or more microfluidic ports on the microfluidic device.

68. A material library comprising a substrate material further comprising a plurality of domains having polymer brushes formed thereon, and a plurality of polypeptide functional groups immobilized to the polymer brushes at one or more of the domains.

69. A material library comprising a substrate material further comprising a plurality of domains having polymer brushes formed thereon, and a plurality of cells immobilized to the polymer brushes at one or more of the domains.

70. The material library of claim 69, wherein the cells are human cells.

71. The material library of claim 69, wherein the cells are human cancer cells.

72. The material library of claim 69, wherein the cells are virally infected cells.

73. The material library of claim 69, wherein the cells are viral cells.

74. The material library of claim 69, wherein the cells are bacterial cells.

75. The material library of claim 74, wherein the bacterial cells are pathogenic strains.

76. A material library comprising a substrate material further comprising a plurality of domains having polymer brushes formed thereon, and one or more types of immunoglobulin molecules immobilized to the polymer brushes at one or more of the domains.

77. The material library of claim 76, wherein the immunoglobulin concentration varies at each domain from about 0.1 fg/mm² antibody immobilized per domain surface area to about 100 mg/mm² antibody immobilized per domain surface area.

78. A material library comprising a substrate material further comprising a plurality of domains having polymer brushes formed thereon, and polypeptide functional groups immobilized to the polymer brushes at one or more of the domains, the polypeptide functional groups capable of interacting with one or more targets.

79. The material library of claim 78, wherein the polypeptide functional groups comprise random polypeptide sequences.

80. The material library of claim 79, wherein the random polypeptide sequences are at least 6-mer sequences.
